(19) United States
(12) Patent Application Publication
(10) Pub. No.: US 2026/0108190 A1
(43) Pub. Date: Apr. 23, 2026

(54) SYSTEMS, DEVICES, AND METHODS FOR PERFORMING TRANSABDOMINAL FETAL OXIMETRY AND/OR TRANSABDOMINAL FETAL PULSE OXIMETRY USING TWO TYPES OF OPTICAL MEASUREMENTS MODALITIES

(71) Applicant: Raydiant Oximetry, Inc., San Ramon, CA (US)

(72) Inventors: Neil Padharia Ray, Sacramento, CA (US); Adam Jacobs, Hollis, NH (US); Rodney P. Chin, Rodeo, CA (US); Tomasz Momot, Bedford, NH (US); Eric Johansson, Livermore, CA (US); Paul Stetson, Piedmont, CA (US); Martin Debreczeny, Berkeley, CA (US)

(21) Appl. No.: 19/117,067

(22) PCT Filed: Oct. 11, 2023
(86) PCT No.: PCT/US2023/034941

§ 371 Date: Mar. 30, 2025

Related U.S. Application Data
(60) Provisional application No. 63/415,258, filed on Oct. 11, 2022.

(51) Int. Cl. A61B 5/1464; A61B 5/00; A61B 5/1455

(52) U.S. Cl. CPC A61B 5/1464; A61B 5/14552; A61B 5/4362; A61B 5/6823; A61B 5/6833; A61B 5/742; A61B 2503/02; A61B 2560/0462; A61B 2562/0233; A61B 2562/146; A61B 2562/185

(57) ABSTRACT

Fetal oximetry information may be determined using a plurality of different types of measurement systems and/or calculation techniques. In some instances, a first measurement system may be used to interrogate maternal abdominal tissue to determine one or more characteristics thereof. Then, a second measurement system may be used to interrogate the pregnant mammal's abdominal tissue as well as fetal tissue and the characteristics of the maternal tissue determined using measurements of the first system may be used to process the measurements obtained using the second system to, for example, increase the accuracy of the processing of the measurements from the second system.

### RELATED APPLICATION

This application is an INTERNATIONAL APPLICATION (PCT) claiming priority to U.S. Provisional Patent Application No. 63/415,258 filed on 11 Oct. 2022 and entitled “SYSTEMS, DEVICES, AND METHODS FOR PERFORMING TRANSABDOMINAL FETAL OXIMETRY AND/OR TRANSABDOMINAL FETAL PULSE OXIMETRY USING A MULTI-FUNCTION TRANSABDOMINAL FETAL OXIMETRY PROBE,” which is incorporated by reference, in its entirety, herein.

### FIELD OF INVENTION

The present invention is in the field of medical devices, oximetry, pulse oximetry, and transabdominal fetal oximetry.

### BACKGROUND

Current methods of monitoring fetal health, such as monitoring fetal heart rate, are inefficient and prone to inaccuracies when determining levels of fetal distress and, at times, provide false positive results indicating fetal distress that may result in the unnecessary performance of a Cesarean delivery. One area of interest in improving fetal health monitoring includes the use of transabdominal fetal oximetry.

Oximetry is a method for determining a level of oxygen saturation of a mammal's tissue, arterial hemoglobin, and/or venous hemoglobin. A mammal's level of oxygen saturation may provide an indication of health or overall wellness of an individual. Transabdominal-fetal-oximetry is a method of oximetry for a fetus performed by analyzing light projected into a pregnant mammal's abdomen that reflects off the fetus contained therein and is detected by a photodetector. The optical information detected by the photodetector is analyzed to calculate fetal oximetry values that may be used to determine whether or not a fetus is in distress and/or is at risk of developing hypoxemia or hypoxia.

### SUMMARY

Systems, devices, and methods for transabdominally determining fetal oximetry information may receive one or more time resolved signal(s) corresponding to light emanating from a pregnant mammal's abdomen. In some embodiments, the time resolved sensor system may interrogate maternal tissue through which light emitted by the continuous wave measurement device passes so that, for example, the pregnant mammal's abdominal tissue characterized by the time resolved signals is the same tissue through which the continuous wave signals pass.

The time resolved signals may be, for example, a frequency domain signal and/or a time-of-flight signal and may be respectively received from a frequency domain sensor system and/or a time-of-flight sensor system. In some embodiments, the time resolved signal may correspond to light of a wavelength greater than 805nm and light of a wavelength less than 805nm.

A characteristic of the pregnant mammal's abdominal tissue and/or the fetus may be determined using the received time resolved signal. Additionally, or alternatively, a measurement of a depth of the fetus at a position corresponding to a component of the continuous wave sensor system may be received.

One or more continuous wave signals corresponding to light traveling through pregnant mammal's abdomen and fetus may also be received from, for example, a continuous wave measurement device. The one or more continuous wave signals may include a first continuous wave signal that corresponds to light of a wavelength greater than 805nm and a second continuous wave signal that corresponds to light of a wavelength less than 805nm.

The one or more continuous wave signals, the characteristic of the pregnant mammal's tissue, and/or the fetal depth may be used to determine fetal oximetry information and the fetal oximetry information may be provided and/or communicated to a display device for display to, for example, a clinician and/or the pregnant mammal. The fetal oximetry information may be, for example, a fetal hemoglobin oxygen saturation level and a fetal tissue oxygenation level.

In some embodiments, a position of the time resolved sensor system on the pregnant mammal's abdomen when the light emanates from the pregnant mammal's abdomen and is detected by the time resolved sensor system is proximate to, or the same as, a position of the continuous wave measurement device on the pregnant mammal's abdomen when the light emanates from the pregnant mammal's abdomen and is detected by the continuous wave measurement device.

Also disclosed herein are patient interfaces configured to cooperate with a transabdominal sensor configured to optically interrogate tissue of a pregnant mammal's abdomen (e.g., maternal abdominal tissue and, in some embodiments, fetal tissue) to determine a characteristic (e.g., light scattering and/or absorption characteristics) of the fetal and/or maternal abdominal tissue. The transabdominal sensor may include one or more light source(s) and a plurality of detectors, each detector of the plurality of detectors including a light detecting surface. The transabdominal sensor may be a frequency domain sensor system, a time-of-flight sensor system, a continuous wave sensor system, a transabdominal fetal oximetry sensor system, and a multi-function transabdominal fetal oximetry sensor. The transabdominal sensor may be configured to transabdominally obtain optical signals that may be processed to determine transabdominal fetal oximetry information.

In some embodiments, the patient interface may be configured to cooperate with the transabdominal sensor by being removably affixed thereto. For example, the patient interface may be configured to be removably attached to transabdominal sensor and to remain on the pregnant mammal's abdomen when the transabdominal sensor may be removed from the patient interface.

The sheet of flexible material may be configured to form a tight optical couple between a pregnant mammal's abdomen and the transabdominal sensor and block ambient light from the environment from entering the pregnant mammal's abdomen and, on most occasions, may be opaque. Additionally, or alternatively, the sheet of flexible material may be configured to prevent shunting light from an optical source of the transabdominal sensor directly to a detector of the transabdominal sensor without passing through tissue of a pregnant mammal's abdomen on which the transabdominal sensor is positioned.

The sheet of flexible material may include a plurality of detector openings that are sized and positioned within the sheet of flexible material to correspond to a size and position of a light detecting surface of a respective one of the plurality of detectors. The sheet of flexible material may also include a light source opening sized, configured, and positioned to correspond to a size and position of the light source. The patient interface may include an adhesive configured to stick to a pregnant mammal's abdomen and/or the transabdominal sensor.

In some embodiments, the patient interface may include a ring configured to align with an outer edge of the patient interface and fit over an outer edge of a patient-facing side of the transabdominal sensor, thereby preventing ambient light from entering a side of the transabdominal sensor when the transabdominal sensor may be positioned on a pregnant mammal's abdomen.

One or more of the detector openings may be covered with a transparent material in a watertight manner. Additionally, or alternatively, one or more of the detector openings may be covered with a material that filters light of undesired wavelength (e.g., ambient and/or visible light).

In some embodiments, the patient interface further includes an optical blanket configured to cover a portion of the pregnant mammal's abdomen proximate to the patient interface and prevent ambient light from entering the covered portion of the pregnant mammal's abdomen. The optical blanket may include an opening sized, shaped, arranged, and configured to cooperate with the patient interface. Additionally, or alternatively, the optical blanket may include an attachment mechanism configured to attach to the transabdominal sensor.

The patient interface may be used by, for example, affixing a patient interface to an epidermis of a pregnant mammal's abdomen above a fetus positioned within, the patient interface including a sheet of flexible material that includes a light source opening and a plurality of detector openings. Then, a transabdominal sensor that includes a light source and a plurality of detectors may be positioned proximate to the patient interface so that the light source aligns with the light source opening in the patient interface and each of the plurality of detectors aligns with a respective detector window of the patient interface. Then, the transabdominal sensor may be attached to the patient interface.

### BRIEF DESCRIPTION OF THE FIGURES

The present invention is illustrated by way of example, and not limitation, in the figures of the accompanying drawings in which:

FIG. 1A is a block diagram of exemplary components that may be included in an exemplary frequency domain sensor system, consistent with some embodiments of the present invention;

FIG. 1B is a block diagram of a patient-facing surface of the frequency domain sensor system of FIG. 1A, consistent with some embodiments of the present invention;

FIG. 2A is a block diagram of exemplary components that may be included in a time-of-flight sensor system, consistent with some embodiments of the present invention;

FIG. 2B is a block diagram of a patient-facing surface of the time-of-flight sensor system of FIG. 2A, consistent with some embodiments of the present invention;

FIG. 3A is a block diagram of exemplary components that may be included in a continuous wave sensor system, consistent with some embodiments of the present invention;

FIG. 3B is a block diagram of a patient-facing surface of the continuous wave sensor system of FIG. 3A, consistent with some embodiments of the present invention;

FIG. 3C is a side view of the frequency domain sensor system of FIG. 1B, the time-of-flight sensor system of FIG. 2B, or the continuous wave sensor system of FIG. 3B, showing a track therein, consistent with some embodiments of the present invention;

FIG. 3D is a side view of the frequency domain sensor system of FIG. 1B, the time-of-flight sensor system of FIG. 2B, or the continuous wave sensor system of FIG. 3B, showing a plurality of attachment mechanisms thereon, consistent with some embodiments of the present invention;

FIG. 4A is a block diagram of an exemplary patient interface, consistent with some embodiments of the present invention;

FIG. 4B provides a block diagram of another exemplary patient interface, consistent with some embodiments of the present invention;

FIG. 5A provides a block diagram of system that includes a first or multi-function transabdominal fetal oximetry sensor with a frequency domain sensor system and a continuous wave sensor system, consistent with some embodiments of the present invention;

FIG. 5B provides a block diagram of system that includes a second multi-function transabdominal fetal oximetry sensor with a time-of-flight sensor system and a continuous wave sensor system, consistent with some embodiments of the present invention;

FIG. 6A provides a top view of a third exemplary multi-function transabdominal fetal oximetry sensor with a swiveling handle in a first orientation, consistent with some embodiments of the present invention;

FIG. 6B provides a top view of the third exemplary multi-function transabdominal fetal oximetry sensor of FIG. 6A with the swiveling handle in a second orientation, consistent with some embodiments of the present invention;

FIG. 6C is a bottom view of the multi-function transabdominal fetal oximetry sensor of FIG. 6A, consistent with some embodiments of the present invention;

FIG. 7A is an exploded view of a first exemplary flexible patient interface assembly, consistent with some embodiments of the present invention;

FIG. 7B is an exploded view of a second exemplary flexible patient interface assembly, consistent with some embodiments of the present invention;

FIG. 8A is a top view is a side view of a system including the multi-function transabdominal fetal oximetry sensor of FIG. 6A seated within a ring of a flexible patient interface assembly, consistent with some embodiments of the present invention;

FIG. 8B is a side view of the system of FIG. 8A, consistent with some embodiments of the present invention;

FIG. 9A provides a bottom view of a fourth exemplary multi-function transabdominal fetal oximetry sensor, consistent with some embodiments of the present invention;

FIG. 9B provides a bottom view of a fifth exemplary multi-function transabdominal fetal oximetry sensor, consistent with some embodiments of the present invention;

FIG. 9C is a side view of the multi-function transabdominal fetal oximetry sensor of FIG. 9A or 9B that includes a track, consistent with some embodiments of the present invention;

FIG. 9D is a side view of the multi-function transabdominal fetal oximetry sensor of FIG. 9A or 9B that includes a plurality of attachment mechanisms, consistent with some embodiments of the present invention;

FIG. 10A is a block diagram of an exemplary patient interface for use with the multi-function transabdominal fetal oximetry sensor of FIG. 9A, consistent with some embodiments of the present invention;

FIG. 10B is a top view of a ring with a center opening configured to accept insertion of a sensor system, consistent with some embodiments of the present invention;

FIG. 10C provides a top view of an assembly of the multi-function transabdominal fetal oximetry sensor of FIG. 9A, the patient interface of FIG. 10A, and the ring, consistent with some embodiments of the present invention;

FIG. 10D provides a cross-section view of the assembly of FIG. 10C, consistent with some embodiments of the present invention;

FIG. 10E provides another cross-section view of the assembly of FIG. 10C, consistent with some embodiments of the present invention;

FIG. 11A is a block diagram of an exemplary patient interface for use with the multi-function transabdominal fetal oximetry sensor of FIG. 9B, consistent with some embodiments of the present invention;

FIG. 11B is a diagram of an assembly of the patient interface of FIG. 11A and the ring, consistent with some embodiments of the present invention;

FIG. 12A is a diagram of an exemplary optical blanket, consistent with some embodiments of the present invention;

FIG. 12B is a diagram of the optical blanket of FIG. 12A with a sensor system inserted therein, consistent with some embodiments of the present invention;

FIG. 12C is a diagram of the optical blanket of FIG. 12A with a ring surrounding an opening therein, consistent with some embodiments of the present invention;

FIG. 12D is a diagram of the optical blanket of FIG. 12C with a sensor system inserted within the ring, consistent with some embodiments of the present invention;

FIG. 13 provides a block diagram of an exemplary system 1300 for executing one or more methods disclosed herein, consistent with some embodiments of the present invention;

FIG. 14 provides a flowchart illustrating a process for using a frequency domain sensor system and a continuous wave sensor system to obtain frequency domain and continuous wave optical measurements of a pregnant mammal's abdomen and a fetus contained within the pregnant mammal's abdomen, consistent with some embodiments of the present invention;

FIG. 15 provides a flowchart illustrating a process for using a time-of-flight sensor system and a continuous wave sensor system to obtain time-of-flight and continuous wave measurements of a pregnant mammal's abdomen and a fetus contained within the pregnant mammal's abdomen, consistent with some embodiments of the present invention;

FIG. 16 provides a flowchart illustrating a process for using a patient interface and a multi-function transabdominal fetal oximetry sensor to obtain one or more transabdominal measurements of a pregnant mammal's abdomen and a fetus contained within the pregnant mammal's abdomen, consistent with some embodiments of the present invention; and

FIG. 17 provides a flowchart illustrating a process for determining fetal oximetry information using measurements from one or more of the frequency domain sensor system, continuous wave sensor system, and the multi-function transabdominal fetal oximetry sensor, consistent with some embodiments of the present invention.

Throughout the drawings, the same reference numerals, and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

Disclosed herein are systems, devices, and methods for obtaining and/or using two or more types of measurements and/or two or more types of analysis of optical signals emanating from a pregnant mammal's abdomen to determine fetal oximetry information. In some instances, a first, or time-resolved, system, device, and/or method may be used to determine optical properties of maternal tissue between a light source and detector receiving light from the light source that has traveled through the maternal tissue. Exemplary time-resolved, or first, systems and/or devices include a frequency domain sensor system and a time-of-flight sensor system. Exemplary optical properties of the maternal and/or fetal tissue that may be determined signals received from the first system and/or device include, but are not limited to, a degree of light scattering (e.g., a scattering coefficient), a degree of light absorption (e.g., absorption coefficient), and a depth of the pregnant mammal's abdominal tissue in the region being exposed to the interrogating light. These maternal optical properties may then be used in the analysis of one or more signals generated by a second system (e.g., a continuous wave sensor system) that include an indication of light that passes through and/or interacts with the pregnant mammal and fetus to, for example, remove portions of the one or more signals that were only incident on the pregnant mammal (i.e., not incident on the fetus) and/or isolate and/or amplify portions of the signal(s) that were incident upon the fetus so that, for example, these resulting signals may be analyzed to determine fetal oximetry information in the form of, for example, fetal hemoglobin oxygen saturation levels and/or fetal tissue oxygen saturation. In some embodiments, the first and second systems may be in separate housings that may be sequentially used to obtain measurements from the same location on the pregnant mammal's abdomen. Alternatively, the first and second systems may reside in the same housing as, for example, a multi-function transabdominal fetal oximetry sensor. Because optical properties of maternal and/or fetal tissue may vary across the pregnant mammal's abdomen/fetus' body, it is advantageous to use the first and second systems/devices in the same or proximate position on the pregnant mammal's abdomen to ensure that the maternal and/or fetal optical properties are consistent for both systems/devices.

Some embodiments of the present invention include a multi-function transabdominal fetal oximetry sensor configured with two or three systems: one to take measurements of fetal oximetry in the frequency domain and/or measure a time-of-flight for photons projected into the pregnant mammal's abdomen and emanating therefrom and the other to take measurements that penetrate the maternal layers of tissue to reach the fetus via, for example, short and long separation optical signals/channels. Having both a frequency system and a system that uses short and long separation optical signals/channels within the same device provides numerous advantages, which include the ability for one system to, for example, assess optical conditions (e.g., scattering, absorption, etc.) at a particular location on the body, such as a pregnant mammal's abdomen, which may then be used by the other system. Because the two systems are closely positioned to one another, a determination of an optical characteristic for one of the systems/locations on the multi-function transabdominal fetal oximetry sensor may be similar to the same optical characteristic at position of the second system due to the relative proximity on the pregnant mammal's abdomen. This may be helpful when performing transabdominal fetal oximetry because a location on the pregnant mammal's abdomen corresponds to a location on the fetus, which may be indicative of, for example, whether the fetal blood that is analyzed for oximetry purposes is pre-ductal or post-ductal blood.

The systems, devices, and methods disclosed herein may be configured to form a tight optical coupling between a sensor system and the pregnant mammal's abdomen to, for example, reduce ambient light that may enter the pregnant mammal's abdomen and/or be detected by a detector of a sensor system and/or reduce a likelihood of optical shunting between components of a sensor system (e.g., light traveling directly from a source to a detector without traveling through the pregnant mammal's abdomen). This optical shunting may cause errors in the form of, for example, a variable shift in the measured signals, which may adversely impact, among other things, measurement accuracy and operational efficiency. Exemplary wavelengths of light that may be used by the sensor systems disclosed herein include but are not limited to 600-900nm.

The frequency domain sensor systems and/or components thereof disclosed may modulate and/or facilitate emission of light that is modulated as a sine wave at frequencies of, for example, 10-500 MHz or 50-300 MHz. Resulting signals (e.g., detected light emanating from a pregnant mammal's abdomen) of interest may include phase delay, AC amplitude, and/or DC amplitude. Instrument variability for measurements made by, and/or signals received from, the frequency domain sensor systems and/or components thereof may be based upon a calibration standard with known optical properties is typically used to correct for instrument variability.

The time-of-flight sensor systems and/or components thereof disclosed herein may emit light as a series of light pulses lasting from, for example, 10 fs-10 ns or 1 ps-1 ns in duration and an output signal of this system (or a component thereof) may be a transient measured following each of the light pulses. The transient may be measured using a technique called time correlated single photon counting (TCSPC) that generates a histogram of photons corresponding to the arrival time. The shape of this histogram may be used to determine the optical properties of the medium (i.e., pregnant mammal and/or fetal tissue and/or blood). Instrument variability for measurements made by, and/or signals received from, the time-of-flight sensor systems and/or components thereof may include measuring an Instrument Response Function (IRF). The IRF may account for the width and shape of the light pulse and/or detector response. The IRF may be convolved with a modeled fitting function when fitting to the measured transient.

The continuous wave sensors and systems disclosed herein may be configured to modulate emitted light of two or more wavelengths by, for example, modulating an operation of two or more different light sources (or a single light source emitting light of different wavelengths), wherein each light source emits light of a particular wavelength into a pregnant mammal's abdomen. In many embodiments, the two or more light sources may operate at their own respective, or independent, frequency. Exemplary modulation frequencies may be within a range of, for example, 10 Hz-10 kHz. When light corresponding to the modulated light from these sources emanates from and/or backscatters through the pregnant mammal's abdomen and is detected by one or more detectors (thereby generating one or more signals), the signals may be analyzed and/or separated from one another using the modulation frequency for the respective wavelength and/or light source.

Additionally, or alternatively, in some embodiments of a continuous wave sensor system, emission of light of different wavelengths (from a single or multiple sources) may be time multiplexed so that, for example, light of each different wavelength is emitted a different time (e.g., in a different time slot) at a known frequency (e.g., 10 Hz-10 kHz). In one example, a continuous wave sensor system may be configured to emit light of a first wavelength (from a first light source), a second wavelength (from a second light source), a third wavelength (from a third light source), and a fourth wavelength (from a fourth light source). The light emitted from the first, second, third, and fourth light sources may be modulated at a known frequency and may also be time multiplexed so that each of the first, second, third, and fourth light sources only emits light at a particular time (e.g., a 2 millisecond time slot each in sequential rotation). In some embodiments, the continuous wave sensor systems disclosed herein may also have an off-duty cycle wherein there is a period in the time multiplexing (e.g., a 2 ms timeslot) wherein no light is emitted. Signals obtained during this time may be analyzed to, for example, measure ambient light and/or other noise that may be impacting the clarity of the signals received resulting from emission (and detection) of light from the first, second, third, and fourth light sources. Continuing with this example, an operation of a continuous wave sensor may establish five timeslots (of 2 ms each) within a 10 ms time period wherein light is emitted from each of the emitted the first, second, third, and fourth light sources for 2 ms each (totally 8 ms) and then no light is emitted during the final 2 ms time slot. The modulated and time multiplexed signals may then be separated from one another using the modulation frequency and time multiplexing routine. Then, light detected by one or more detectors of the continuous wave system may be analyzed by separating out light corresponding to each wavelength using the modulation rate (i.e., 100 Hz) and time multiplexing routine (e.g., 2 ms time slots for each wavelength of light in sequence) and these separate signals may be further analyzed to, for example, determine fetal oxygenation information.

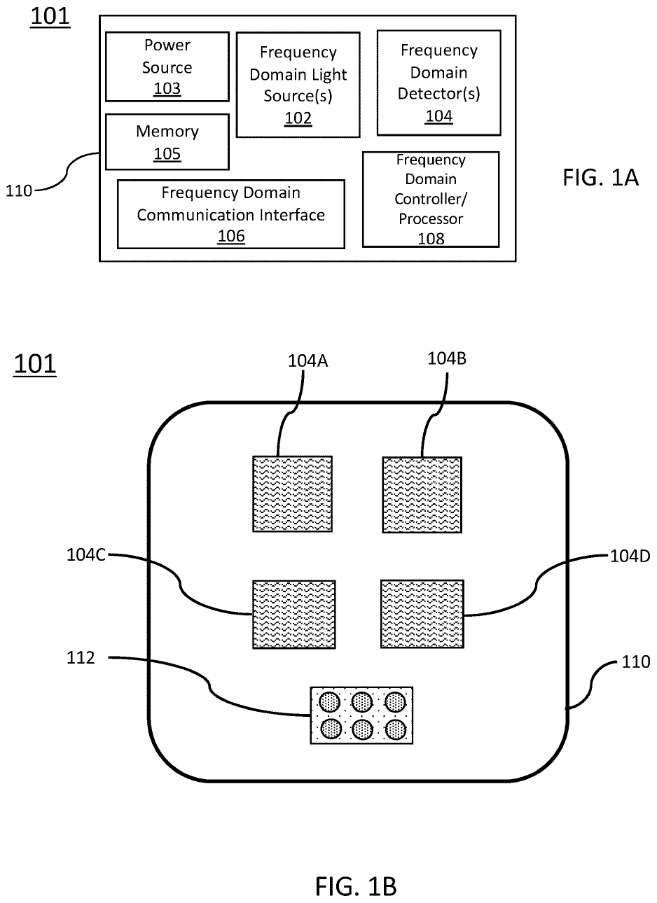

Turning now to the figures, FIG. 1A is a block diagram of exemplary components that may be included in an exemplary first system in the form of a frequency domain sensor system 101 that may be used to, for example, interrogate maternal abdominal tissue and/or fetal tissue to determine one or more characteristics thereof as, for example, described herein. Frequency domain sensor system 101 may be configured to communicate with, cooperate with, and/or receive instructions from a controller system like controller system 540 and/or a computer like computer 1310 as, for example, disclosed herein.

Frequency domain sensor system 101 includes a housing 110 in which one or more frequency domain light source(s) and one or more frequency domain detectors 104 are resident. Components of frequency domain sensor system 101 may be configured to obtain and/or generate optical measurements of maternal and, in some cases, fetal tissue that are compatible with frequency domain analysis. In some embodiments, frequency domain sensor system 101 and/or a component thereof may be a part of and/or be communicatively and/or electrically coupled to a frequency-sensitive and/or phase-sensitive spectrometer. Exemplary frequency domain sensor systems 101 include, but are not limited to, the OXIPLEXTS™ and/or the METAOX™ tools manufactured by ISS are examples.

Frequency domain light source(s) 102 may be any device, conduit, and/or optical coupling configured and arranged to emit light and/or facilitate the emission and/or direction of light of one or more wavelengths into a maternal abdomen and/or fetus contained therein. Exemplary frequency domain light sources 102 include, but are not limited to, light emitting diodes (LEDs) and/or an optical coupling. When frequency domain light source(s) 102 are embodied as optical couplings, these optical couplings may be configured to optically and physically couple to an optical fiber that may be coupled to a centralized light source (e.g., a laser and/or a plurality of lasers) and, once coupled to the centralized light source, the frequency domain light source(s) 102 are embodied as optical couplings may be configured to allow the light from the centralized light source to pass therethrough and exit frequency domain sensor system 101 (e.g., be projected into a pregnant mammal's abdomen. The centralized light source may emit light that is carried by the optical fiber to the frequency domain light source(s) 102, which in these embodiments, are instantized as optical couplings, and light may exit housing 110 via these optical couplings. Exemplary wavelengths for light emitted by frequency domain light source(s) 102 may be within a range of 600-1200 nm or within a range of 700-900 nm. Often times, frequency domain light source(s) 102 may be configured to emit light of at least 2 wavelengths, one above and one below the hemoglobin isosbestic point (i.e., approximately 805 nm) and, in some exemplary embodiments, the frequency domain light source(s) may emit light of 730 and 850 nm.

Emission of light by frequency domain light source(s) 102 may be modulated according to one or more frequencies. Exemplary modulation frequencies may be within the range of 10-500MHz or 50-300 MHz. Light emitted by frequency domain light source(s) 102 may travel through the pregnant mammal's abdomen and, in some cases, be incident on the fetus therein, and may then be reflected and/or backscattered through the pregnant mammal's abdomen whereupon light emitted from the pregnant mammal's abdomen may be detected by one or more frequency domain detectors 104.

Frequency domain detectors 104 may be, for example, photodetectors configured to convert an optical signal (e.g., photons originally emitted by one or more frequency domain light source(s) 102 and have traveled through the pregnant mammal's abdomen) into an electronic and/or analog signal. Additionally, or alternatively, frequency domain detectors 104 may be an optical coupling configured to physically and optically couple to a first end of an optical cable, wherein a second end of the optical cable is optically coupled to a photodetector or other device that may convert an optical signal traveling through a frequency domain detector 104 and an optical fiber coupled thereto into an electronic and/or analog signal. In some embodiments, gains of one or more of frequency domain detector(s) 104 may be modulated at a similar, or the same, frequency as modulation of frequency domain light source(s) 102. For example, when a modulation frequency is 110 MHz for a frequency domain light source 104, gains of one or more of the frequency domain detector(s) 104 may be modulated at 110 MHz+1 KHz so that the detector signal at the difference frequency of 1 kHz may be detected. This is known as heterodyne detection. One or more signals detected by frequency domain detector(s) 104 may be analyzed and/or separated from one another using, for example, the modulation frequency of the frequency domain light source(s) 102 and/or a modulation frequency of gains of the frequency domain detector(s) 104. This analysis and/or signal separation may be performed by, for example, a processor and/or computer such as the processors and/or computers disclosed herein.

In some instances, frequency domain light source(s) 102 may be configured to multiplex between different frequencies and/or wavelengths of light at, for example, pre-determined intervals so that, for example, corresponding detected optical signals of different wavelengths and/or different modulation frequencies may be In some embodiments, housing 110 may also house an optional power source 103, an optional memory 105, an optional frequency domain communication interface 106, and/or an optional frequency domain controller and/or processor 108. Additionally, or alternatively, these components may reside outside of housing 110 in, for example, an external system or computer. Optional power source 103 may be any source of electrical power for one or more components of the frequency domain sensor system 101. Exemplary power sources 103 include, but are not limited to, batteries, AC/DC converters, and mechanisms to couple to an electrical main power source (e.g., electrical plug connector and/or an AC/DC converter). In some embodiments, power source 103 may include and/or be coupled to an isolation means.

Optional memory 105 may be configured to store one or more sets of instructions for operating frequency domain sensor system 101 and/or components of system 101 according to, for example, one or more processes disclosed herein. Additionally, or alternatively, memory 105 may be configured to store information collected, measured, and/or determined by components of frequency domain sensor system 101 while, for example, executing one or more processes disclosed herein. Frequency domain controller/processor 108 may be configured to execute one or more processes disclosed herein and/or control an operation (e.g., turn off, turn on, initiate communication, multiplex operations of different frequency domain light sources 102, etc.) of one or more components of frequency domain sensor system 101 such as one or more frequency domain light source(s) 102 and/or frequency domain detector(s) 104. In some embodiments, frequency domain controller/processor 108 may be configured to detect, process, pre-process, filter (e.g., band pass, noise reduction, etc.), and/or amplify a signal received from one or more frequency domain detector(s) 104 so that, for example, signal-to-noise ratio may be improved and/or signal size may be reduced and/or compressed.

Optional communication interface 106 may be configured to receive data from and/or communicate data to a user and/or an external component such as the external components disclosed herein (e.g., continuous wave sensor system 301, controller system 540, tissue oxygenation system 530, and/or computer and/or processor 1310 as, for example, discussed below). Exemplary data communicated by communication interface 106 includes, but is not limited to, data gathered by frequency domain detectors 104, an error message, a duty cycle, and/or an operation performed by frequency domain sensor system 101. Exemplary data received by communication interface 106 includes sets of instructions for the operation of components of frequency domain sensor system 101 that may be consistent with one or more processes described herein. Communication interface 106 may be any interface (e.g., wired, ethernet, wireless, Wi-Fi, etc.) configured to allow communication interface 106 to receive and/or communicate data to and/or from frequency domain sensor system 101. In some embodiments, communication interface 106 may be a port (e.g., USB, Ethernet, wireless communication port, etc.) into which an external component may be coupled. Additionally, or alternatively, communication interface 106 may be configured to accept input from one or more users as, for example, a keypad, keyboard, speaker, microphone, and/or touch-sensitive display.

FIG. 1B is a block diagram of a patient-facing surface of frequency domain sensor system 101 and shows an exemplary arrangement of frequency domain light source(s) 102 in the form of light source array 112 that includes six frequency domain light sources 102 (which may be embodied as, for example, LEDs and/or optical couplings configured to couple to an external light source via an optical fiber) and an array of four frequency domain detectors 104A, 104B, 104C, and 104D arranged in two rows of two columns as shown within housing 110 so that, for example, different source/detector distances across frequency domain sensor system 101 may capture light traveling along optical paths of varying depths (e.g., short separation and/or long separation measurements) and/or geometries following projection into and/or backscattering from a pregnant mammal's abdomen and/or a fetus therein as, for example, disclosed herein. For example, a distance between third and fourth frequency domain detectors 104C and 104D may be within a range of 1-4 cm or 2-3 cm so that, for example, light traveling a relatively shallow path through of the pregnant mammal's abdomen may be detected by third and fourth frequency domain detectors 104C and 104D. A distance between first and second frequency domain detectors 104A and 104B may be within a range of 6-9 cm or 6-7 cm so that, for example, light traveling a relatively deeper path through of the pregnant mammal's abdomen and potentially the fetus may be detected by first and second frequency domain detectors 104A and 104B. Stated differently, the different source/detector distances across frequency domain sensor system 101 may capture light traveling along optical paths of varying depths (e.g., short separation (e.g., only traveling through maternal tissue) and/or long separation measurements (e.g., traveling through maternal and fetal tissue)) and/or geometries following projection into and backscattering from different depths of a pregnant mammal's abdomen and/or a fetus therein as, for example, disclosed herein.

In some instances, a sensitivity, or size, of first, second, third, and fourth frequency domain detectors 104A-104D may vary relative to one another so that, for example, a third frequency domain detector 104C and a fourth frequency domain detector 104D, which are both physically the closest to frequency domain light array 112, may be less sensitive and/or smaller when compared to a first frequency domain detector 104A and a second frequency domain detector 104B, which are further away from frequency domain light array 112. A greater size and/or sensitivity for first frequency domain detector 104A and second frequency domain detector 104B may be desirable so that, for example, first frequency domain detector 104A and second frequency domain detector 104B are able to detect optical signals that have a longer path length (e.g., an emission position further from frequency domain light array 112 than an emission position for light detected by third or fourth frequency domain detectors 104C and 104D) and may, therefore, be relatively weaker than optical signals detected by third or fourth frequency domain detectors 104C and 104D.

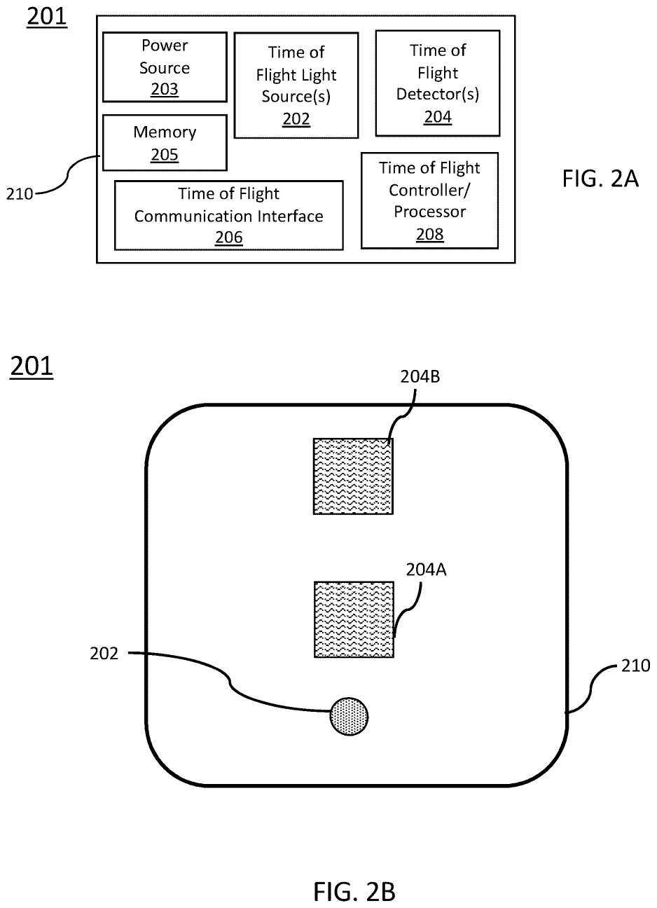

FIG. 2A is a block diagram of exemplary components that may be included in an exemplary time-of-flight sensor system 201 as disclosed and used herein to, for example, interrogate maternal abdominal tissue and/or fetal tissue to determine one or more characteristics thereof as, for example, described herein. In some embodiments, time-of-flight sensor system 201 may be configured to measure tissue oxygen saturation.

Time-of-flight sensor system 201 may be configured to communicate with, cooperate with, and/or receive instructions from a controller system like controller system 540 and/or a computer like computer 1310 as, for example, disclosed herein. Exemplary time-of-flight sensor systems 201 include, but are not limited to, the PioNIRS system and the Kernel Flow system. Time-of-flight sensor system 201 includes a housing 210 in which one or more time-of-flight light source(s) and one or more time-of-flight detectors 204 are resident.

Time-of-flight light source(s) 202 may be any device, conduit, and/or optical coupling configured and arranged to emit light and/or facilitate the emission of light of one or more wavelengths into a maternal abdomen and time-of-flight detector(s) 204 may be any detector, conduit, and/or optical coupling configured to receive and/or detect light emanating from the maternal abdomen and produce and/or facilitate the production of a signal (digital and/or analog) that provides information about the detected light. Exemplary time-of-flight light sources 202 may be similar to frequency domain light sources 102 in, for example, form and/or function and may include, but are not limited to, light emitting diodes (LEDs) and a laser that may be optically coupled to time-of-flight sensor system 201 via a time-of-flight light source 202 embodied as an optical coupling. Exemplary wavelengths for light emitted by time-of-flight light source(s) 202 may be within a range of 600-1200 nm or within a range of 700-900 nm. Often times, time-of-flight light source(s) 102 may be configured to emit light of at least 2 wavelengths, one above and one below the hemoglobin isosbestic point (i.e., approximately 805 nm) and, in some exemplary embodiments, the frequency domain light source(s) may emit light of 730 and 850 nm as a series of pulses lasting, for example, 100-0.01 ns in duration.

Exemplary time-of-flight detectors 204 may be similar to frequency domain detectors 102 in, for example, form and/or function and may include, but are not limited to silicon photodiodes, cameras, traditional photomultiplier tubes, silicon photomultipliers, and/or avalanche photodiodes. In some embodiments, time-of-flight detectors 204 may be embodied as an optical coupling to which a first end of an optical fiber may be coupled, with a second end of the optical fiber being coupled to a photodetector that is not resident within housing 210. In these embodiments, time-of-flight detectors 204 may act as a conduit for light emanating from the pregnant mammal's abdomen so that this light may enter the optical fiber and be delivered to the photodetector that is not resident within housing 210.

In some embodiments, time-of-flight sensor system 201 may include an optional power source 203, an optional memory 205, an optional time-of-flight communication interface 206, and/or an optional time-of-flight controller and/or processor 208. Optional memory 205 may be configured to store one or more sets of instructions for operating time-of-flight sensor system 201 and/or components of time-of-flight system 201 according to, for example, one or more processes disclosed herein. Additionally, or alternatively, memory 205 may be configured to store information collected, measured, and/or determined by components of time-of-flight sensor system 201 while, for example, executing one or more processes disclosed herein. Optional time-of-flight controller/processor 208 may be configured to execute one or more processes disclosed herein and/or control an operation (e.g., turn off, turn on, initiate communication, etc.) of one or more components of time-of-flight sensor system 201 such as one or more time-of-flight light source(s) 202 and/or time-of-flight detector(s) 204. In some embodiments, time-of-flight controller/processor 208 may be configured to detect, process, pre-process, filter (e.g., band pass, noise reduction, etc.), and/or simplify a signal received from one or more components of time-of-flight sensor system 201 so that, for example, signal-to-noise ratio may be improved and/or signal size may be reduced and/or compressed.

Optional power source 203 may operate and/or be configured in a manner similar to power source 103. Optional time-of-flight communication interface 206 may include components similar to frequency domain communication interface 106 and may be operable to receive and/or communicate data to and/or from a user and/or an external component such as the external components disclosed herein (e.g., continuous wave sensor system 301, controller system 540, tissue oxygenation system 530, and/or computer and/or processor 1310 as, for example, discussed below). in a manner similar to frequency domain communication interface 106. Exemplary data communicated by communication interface 206 includes, but is not limited to, data gathered by time-of-flight detectors 204. Exemplary data received by communication interface 206 includes sets of instructions for the operation of components of time-of-flight sensor system 201.

FIG. 2B is a block diagram of a patient-facing surface of an exemplary time-of-flight sensor system 201 and shows an exemplary arrangement of a time-of-flight light source 202 and a short-distance time-of-flight detector 204A and a long-distance time-of-flight detector 204B arranged within housing 210. Short-distance time-of-flight detector 204A may be positioned within a range of, for example, 1-4 cm or 2-3 cm from time-of-flight light source 202 and long-distance time-of-flight detector 204B may be positioned within a range of, for example, 4-7 cm or 5-6 cm from time-of-flight light source 202. Due to its longer source-detector distance, long-distance time-of-flight detector 204B is able to detect optical signals that have a longer path length and/or travel deeper into the maternal tissue than short-distance time-of-flight detector 204A.

One exemplary reason why that the frequency domain sensor system 101 has four detectors, while time-of-flight sensor system 201 has only 2 is that performance of frequency domain analysis may involve computing slopes of amplitude and phase of detected frequency domain signals as a function of source-detector distance, wherein the pair of closer frequency domain detectors (i.e., third and fourth frequency domain detectors 104C and 104D) may be used to interrogate the relatively shallow maternal-only tissue and the pair of frequency domain detectors (i.e., first and second frequency domain detectors 104A and 104B) further away from frequency domain light source array 112 may be used to interrogate deeper into the maternal abdomen and/or the fetus, which is possible because of the longer source-detector distance for the pair of frequency domain detectors positioned further away from light source array 112. Algorithms used to calculate time-of-flight measurements may achieve a similar goal by using only two time-of-flight detectors by fitting a shape of a time-of-flight transient curve (e.g., a histogram and/or decay curve) and/or fitting a temporal moment (e.g., first, second, and/or third moments) of the transient to extract optical properties of the detected light.

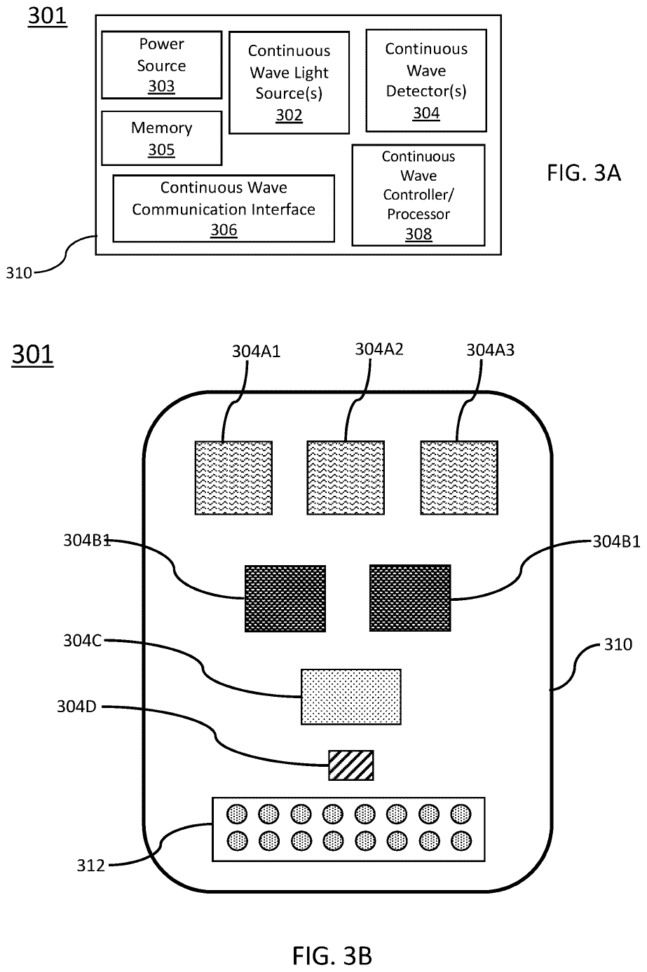

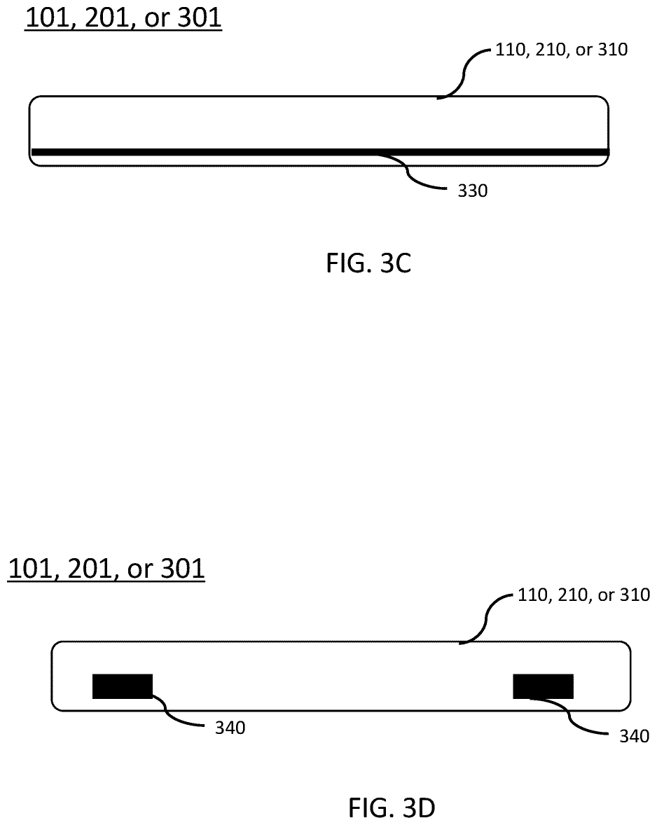

FIG. 3A is a block diagram of exemplary components that may be included in an exemplary continuous wave sensor system 301 as disclosed and used herein to, for example, interrogate maternal abdominal tissue and/or fetal tissue to determine one or more characteristics (e.g., oxygenation levels, light scattering levels, light absorption levels, etc.) thereof and/or fetal oximetry information as, for example, described herein. Continuous wave sensor system 301 includes a housing 310 in which one or more continuous wave light source(s) and one or more continuous wave detectors 304 are resident. On some occasions, continuous wave sensor system 301 may include and/or be communicatively coupled to a spectrometer that provides a means of wavelength-resolving broadband light emissions. Continuous wave sensor system 301 may be configured to communicate with, cooperate with, and/or receive instructions from a controller system like controller system 540 and/or a computer like computer 1310 as, for example, disclosed herein.

Continuous wave light source(s) 302 may be any device, conduit, and/or optical coupling configured and arranged to emit light and/or facilitate the emission of light of one or more wavelengths into a maternal abdomen and continuous wave detector(s) 304 may be any detector, conduit, and/or optical coupling configured to receive and/or detect light emanating from the maternal abdomen and produce and/or facilitate the production of a signal (digital and/or analog) that provides information about the detected light. Exemplary continuous wave light sources 302 include, but are not limited to, light emitting diodes (LEDs) and lasers that may be optically coupled to continuous wave sensor system 301 when continuous wave light sources 302 are embodied as optical couplings. Exemplary continuous wave detectors 304 include, but are not limited to, silicon photodiodes, cameras, traditional photomultiplier tubes, silicon photomultipliers, and/or avalanche photodiodes.

Optionally, continuous wave sensor system 301 may include one or more of a power source 303, a memory 305, a continuous wave communication interface 306, and a continuous wave controller and/or processor 308 resident within housing 310. Optional power source 303 may operate and/or be configured in a manner similar to power source(s) 103 and/or 203. Optional continuous wave communication interface 306 may include components similar to frequency domain communication interface 106 and/or time-of-flight communication interface 206 and may be operable to receive and/or communicate data to and/or from a user to and/or an external component such as the external components disclosed herein (e.g., controller system 540, tissue oxygenation system 530, and/or computer and/or processor 1310 as, for example, discussed below). in a manner similar to frequency domain communication interface 106 and/or time-of-flight communication interface 206. Exemplary data communicated by communication interface 306 includes, but is not limited to, data gathered by continuous wave detectors 304, an error message, and/or operational parameters or functions performed by continuous wave sensor system 301. Exemplary data received by communication interface 306 includes sets of instructions for the operation of components of continuous wave sensor system 301.

Optional memory 305 may be configured to store one or more sets of instructions for operating continuous wave sensor system 301 and/or components of system 301 according to, for example, one or more processes disclosed herein. Additionally, or alternatively, memory 305 may be configured to store information collected, measured, and/or determined by components of continuous wave sensor system 301 while, for example, executing one or more processes disclosed herein. Continuous wave controller/processor 308 may be configured to execute one or more processes disclosed herein and/or control an operation (e.g., turn off, turn on, initiate communication, etc.) of one or more components of continuous wave sensor system 301 such as one or more continuous wave light source(s) 302 and/or continuous wave detector(s) 304. In some embodiments, continuous wave controller/processor 308 may be configured to detect, process, pre-process, filter (e.g., band pass, noise reduction, etc.), and/or amplify a signal received from one or more components of continuous wave sensor system 301 (e.g., detector 304) so that, for example, signal-to-noise ratio may be improved and/or signal size may be reduced and/or compressed.

FIG. 3B is a block diagram of an exemplary patient-facing surface of continuous wave sensor system 301 and shows an exemplary arrangement of continuous wave light source(s) 302 in the form of a light source array 312 that includes sixteen light sources and an array of seven continuous wave detectors 304 that includes a first row of continuous wave detectors 304A1, 304A2, and 304A3 that are positioned to have the largest separation from light source array 312; a second row of two continuous wave detectors 304B1 and 304B2 that are positioned closer to light source array 312 than the first row of continuous wave detectors; a third row that includes one continuous wave detector 304C that is positioned closer to light source array 312 than the second row of detectors; and a fourth row that includes one continuous wave detector 304D that is positioned closest to light source array 312. The different source/detector distances across continuous wave sensor system 301 may capture light traveling along optical paths of varying depths/lengths (e.g., short separation and/or long separation measurements) and geometries (e.g., different angles relative to light source array 312 as light travels through a pregnant mammal's abdomen and/or a fetus therein) as, for example, disclosed herein.

In some instances, a sensitivity, or size, of the continuous wave detectors 304 within housing 310 may vary so that, for example, continuous wave detectors positioned closer to continuous wave light array 312 may be less sensitive and/or smaller when compared to the continuous wave detectors positioned further away from 304A and large continuous wave detectors 304B, which are further away from continuous wave light array 312. A greater size and/or sensitivity for continuous wave detectors 304 may be desirable so that, for example, these continuous wave detectors 304 are able to detect optical signals that have a longer path length and may, therefore, be relatively weaker that optical signals detected by the continuous wave detectors positioned closer to continuous wave light array 312. In another embodiment, continuous wave sensor system 301 may include a single detector (e.g., a 10×10 mm detector) that is configured to operate at a plurality of source-detector distances instead of plurality of different detectors such as first row of continuous wave detectors 304A1, 304A2, and 304A3, and/or second row of continuous wave detectors 304B1 and 304B2 which are positioned further away from the light source (e.g., continuous wave light source array 312).

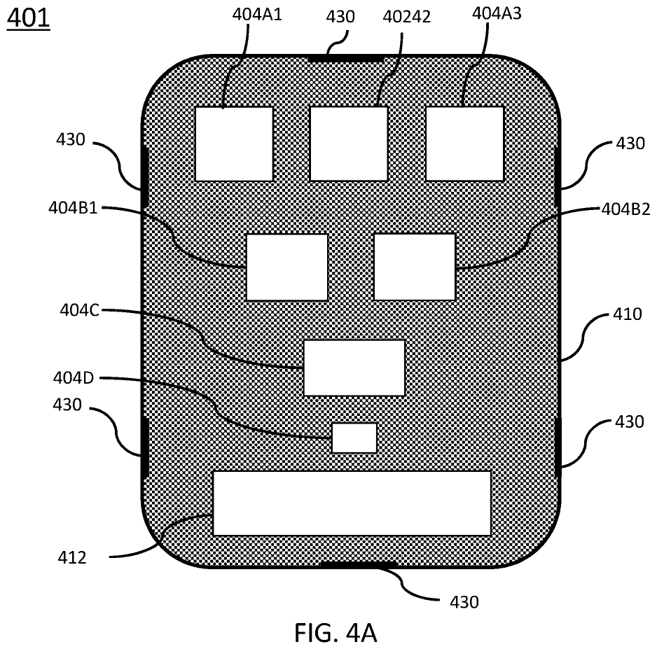

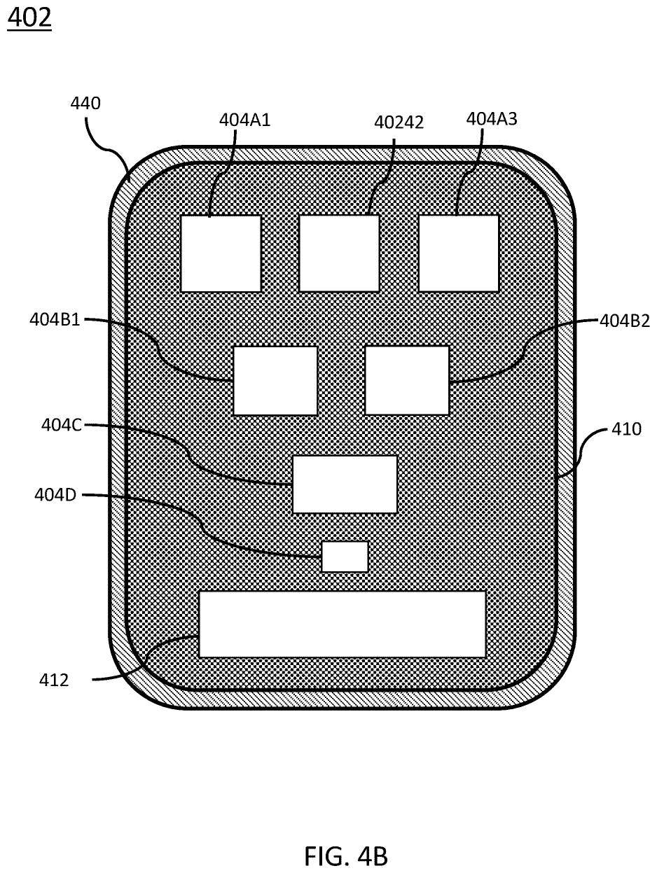

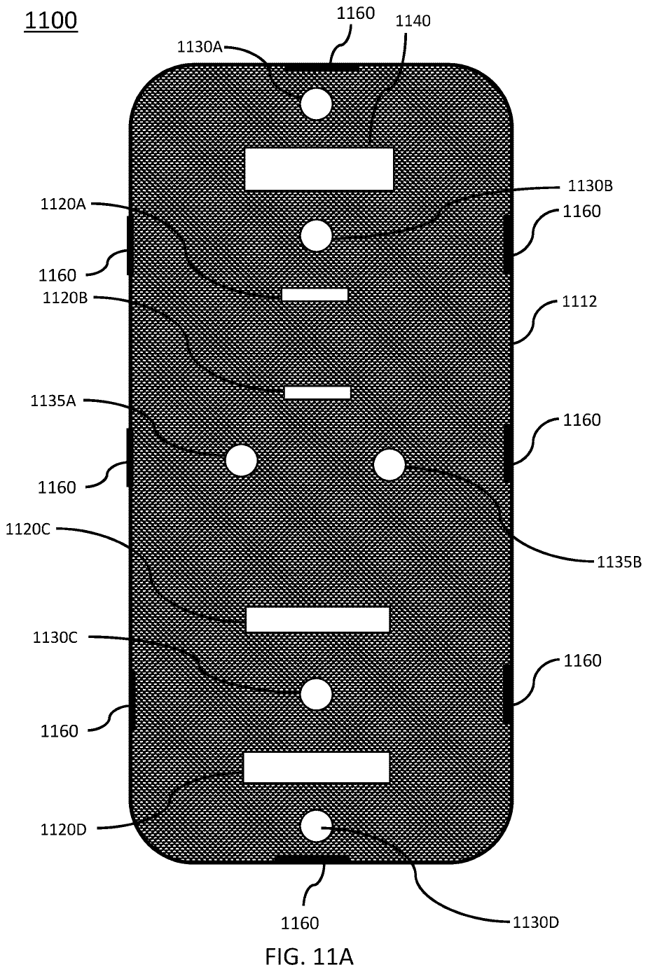

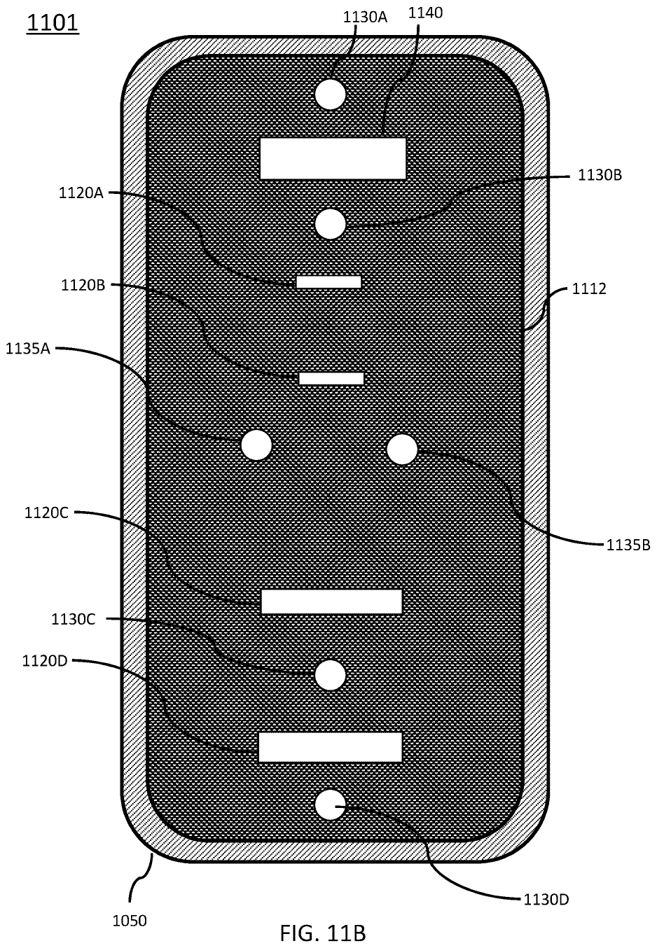

FIG. 3C is a side view of frequency domain sensor system 101, time-of-flight sensor system 201, or continuous wave sensor system 301 in which a track 330 extends along a lower (as oriented in the figure) edge of respective frequency domain sensor system housing 110, time-of-flight sensor system housing 210, or continuous wave sensor system housing 310. Track 330 may be configured to cooperate with one or more attachment mechanisms provided by, for example, a patient interface such as patient interface 401 shown in FIG. 4A and discussed below and/or an optical blanket like optical blanket 1110 shown in FIGS. 11A-11D discussed below. Track 330 may extend around all four sides of frequency domain sensor system 101, time-of-flight sensor system 201, and/or continuous wave sensor system 301 and/or a portion thereof. For example, track 330 may extend along a portion of the sides of frequency domain sensor system 101, time-of-flight sensor system 201, or continuous wave sensor system 301 that correspond to the one or more attachment mechanisms. Additionally, or alternatively, frequency domain sensor system 101, time-of-flight sensor system 201, and/or continuous wave sensor system 301 may include one or more attachment mechanisms 340 (e.g., a clamp, snap, or clip) as shown in FIG. 3D. Attachment mechanisms 340 may be configured to attach frequency domain sensor system 101, time-of-flight sensor system 201, and/or continuous wave sensor system 301 to a patient interface and/or optical blanket as disclosed herein. Attachment mechanisms 340 may be positioned on one or more sides of frequency domain sensor system 101, time-of-flight sensor system 201, and/or continuous wave sensor system 301.

FIG. 4A is a block diagram of an exemplary patient interface 401 for use with continuous wave sensor system 301. Patient interface 401 may include a patient interface sheet 410 (e.g., foam, fabric, plastic, etc.) sized, shaped, and configured to cover a patient-facing surface of continuous wave sensor system 301 without obscuring any components thereof. In some embodiments, a thickness (e.g., 1-10 mm) of patient interface 401 may be known so that it may be factored into, for example, geometrical and/or temporal calculations of light exiting and/or entering continuous wave sensor system 301. Patient interface sheet 410 may include a plurality of windows 404 sized, shaped, and arranged within patient interface 401/patient interface sheet 410 to align with the light sources and/or detectors of continuous wave sensor system 301. For example, patient interface sheet 410 may include a first extra-large detector window 404A1 sized, shaped, and arranged within patient interface sheet 410 to correspond to a position, size, and shape of first extra-large detector 302A1; a second extra-large detector window 404A2 sized, shaped, and arranged within patient interface sheet 410 to correspond to a position, size, and shape of second extra-large detector 302A2; a third extra-large detector window 404A3 sized, shaped, and arranged within patient interface sheet 410 to correspond to a position, size, and shape of first extra-large detector 302A3; a first large detector window 404B1 sized, shaped, and arranged within patient interface sheet 410 to correspond to a position, size, and shape of first large detector 302B1; a second large detector window 404B2 sized, shaped, and arranged within patient interface sheet 410 to correspond to a position, size, and shape of second large detector 302B2; a medium detector window 404C sized, shaped, and arranged within patient interface sheet 410 to correspond to a position, size, and shape of medium detector 304C; a small detector window 404D sized, shaped, and arranged within patient interface sheet 410 to correspond to a position, size, and shape of small detector 304D; and a light array window 412 sized, shaped, and arranged within patient interface sheet 410 to correspond to a position, size, and shape of continuous wave light array 312.

One or more detector window(s) 404 may be open (i.e., a hole in patient interface sheet 410 with no covering) or covered. When covered, exemplary coverings include, but are not limited to, a clear, or transparent material (e.g., acrylic or plastic) and/or a material that filters and/or polarizes incoming and/or outgoing light. For example, one or more coverings for a detector window 404 may be selected and/or configured to filter out visible light and/or light that may be expected to correspond to ambient light in an environment where continuous wave sensor system 301 is being used (e.g., florescent light). Continuous wave sensor system 301 may be coupled to patient interface 401 via, for example, a mechanical device (e.g., a clip or snap) and/or an adhesive. Additionally, or alternatively, continuous wave sensor system 301 may be coupled to patient interface 401 via attachment mechanisms 430 configured, sized, and arranged around a perimeter of patient interface 401 to correspond to and/or cooperate with an attachment mechanism of continuous wave sensor system 301 such as track 330 and/or attachment mechanisms 340. In many embodiments, a patient-facing side of patient interface 401 may be coated with, for example, an adhesive or other compound that may be configured to affix patient interface 401 to a pregnant mammal's skin or epidermis. In some embodiments, a clinician may affix patient interface 401 on the pregnant mammal's abdomen in a location selected to be directly above the fetus or a region of interest of the fetus (e.g., brain or back) so that patient interface 401 remains in this known position. In some embodiments, a patient interface like patient interface 401 may be configured for cooperation with frequency domain sensor system 101 an/d/or time-of-flight sensor system 201 in a manner similar to how patient interface 401 is configured to cooperate with continuous wave sensor system 301.

FIG. 4B provides a block diagram of another exemplary patient interface 402 for use with continuous wave sensor system 301. Patient interface 402 includes a ring 440 that extends in the Z direction by 2-10 mm above a sensor-facing surface of sheet of patient interface sheet 410. Ring 440 is sized, arranged, and/or configured to correspond to a shape of an exterior patient facing portion of continuous wave sensor system housing 310 so that continuous wave sensor system housing 310 may fit into ring 410 and be held next to the sensor-facing surface of sheet of patient interface sheet 410 via, for example, friction, a track, and/or an attachment mechanism. Ring 440 may be configured to optically isolate detectors of continuous wave sensor system 301 from ambient light and/or prevent shunting of light from a continuous wave light source to a continuous wave detector.

In some embodiments, a frequency domain sensor system 101 and continuous wave sensor system 301 may reside in the same housing so that, for example, a position of the frequency domain sensor system 101 and continuous wave sensor system 301 and/or components thereof may be proximate to one another so that, for example, the same maternal/fetal tissue is interrogated by both the frequency domain and continuous wave sensor systems and/or relative positions of components of the frequency domain and continuous wave sensor systems 101 and 301 may be known. These relative positions may be factored into calculations and/or determinations of, for example, maternal and/or fetal optical properties and/or maternal and/or fetal blood and/or tissue oxygenation levels.

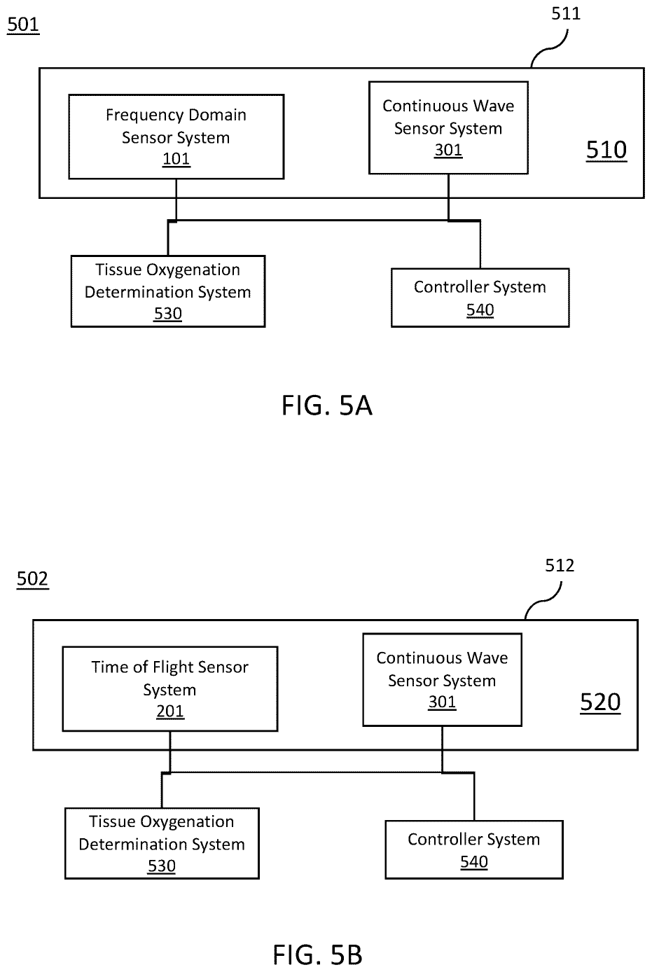

FIG. 5A provides a block diagram of system 501 that includes a first combined, or multi-function, transabdominal fetal oximetry sensor 510 that includes frequency domain sensor system 101 and continuous wave sensor system 301 within a housing 511. First combined, or multi-function, transabdominal fetal oximetry sensor 510 is communicatively coupled to an optional controller system 540 configured to, for example, receive signals from and/or control one or more operations of first combined, or multi-function, transabdominal fetal oximetry sensor 510 and/or a component thereof. For example, controller system 540 may be configured to control modulation (e.g., emitted light and/or detector gains), time multiplexing, and/or a duty cycle for one or more components of first multi-function transabdominal fetal oximetry sensor 510. Additionally, or alternatively, controller system 540 may be configured to control a wavelength of light emitted by a frequency domain light source 102 and/or a continuous wave light source 302.

Optionally, first combined, or multi-function, transabdominal fetal oximetry sensor 510 may be communicatively coupled to an optional tissue oxygenation determination system 530 configured to use outputs from of frequency domain sensor system 101 and/or continuous wave sensor system 301 to characterize maternal and/or fetal tissue and/or determine a level of tissue oxygenation for the maternal and/or fetal tissue according to, for example, one or more processes described herein.

Additionally, or alternatively, in some embodiments, a frequency domain sensor system 101 and continuous wave sensor system 301 may reside in the same housing so that, for example, a position of the frequency domain sensor system 101 and continuous wave sensor system 301 and/or components thereof may be proximate to one another so that, for example, the same maternal/fetal tissue is interrogated by both the frequency domain and continuous wave sensor systems and/or relative positions of components of the frequency domain and continuous wave sensor systems 101 and 301 may be known. These relative positions may be factored into calculations and/or determinations of, for example, maternal and/or fetal optical properties and/or maternal and/or fetal blood and/or tissue oxygenation levels.

FIG. 5B provides a block diagram of a system 502 that includes a second combined, or multi-function, transabdominal fetal oximetry sensor 520 that includes time-of-flight sensor system 201 and continuous wave sensor system 301 within a housing 512. Second combined, or multi-function, transabdominal fetal oximetry sensor 520 may be optionally communicatively coupled to controller system 540, which may be configured to, for example, receive signals from and/or control one or more operations of second combined, or multi-function, transabdominal fetal oximetry sensor 520 and/or a component thereof. For example, controller system 540 may be configured to control light pulses from time-of-flight sensor system 201, time multiplexing for continuous wave sensor system 301, and/or a duty cycle for one or more components of second multi-function transabdominal fetal oximetry sensor 520. Additionally, or alternatively, controller system 540 may be configured to control a wavelength of light emitted by a time-of-flight light source 202 and/or a continuous wave light source 302,

Second combined, or multi-function, transabdominal fetal oximetry sensor 520 may be communicatively coupled to an optional tissue oxygenation determination system 530 configured to use outputs from of time-of-flight sensor system 201 and/or continuous wave sensor system 301 to characterize maternal and/or fetal tissue and/or determine a level of tissue oxygenation for the maternal and/or fetal tissue according to, for example, one or more processes described herein.

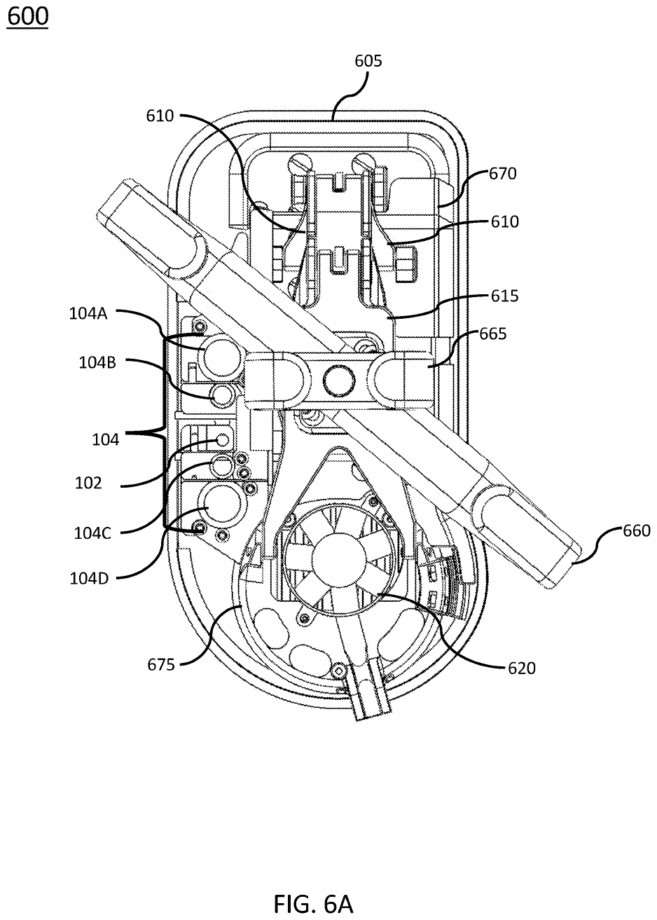

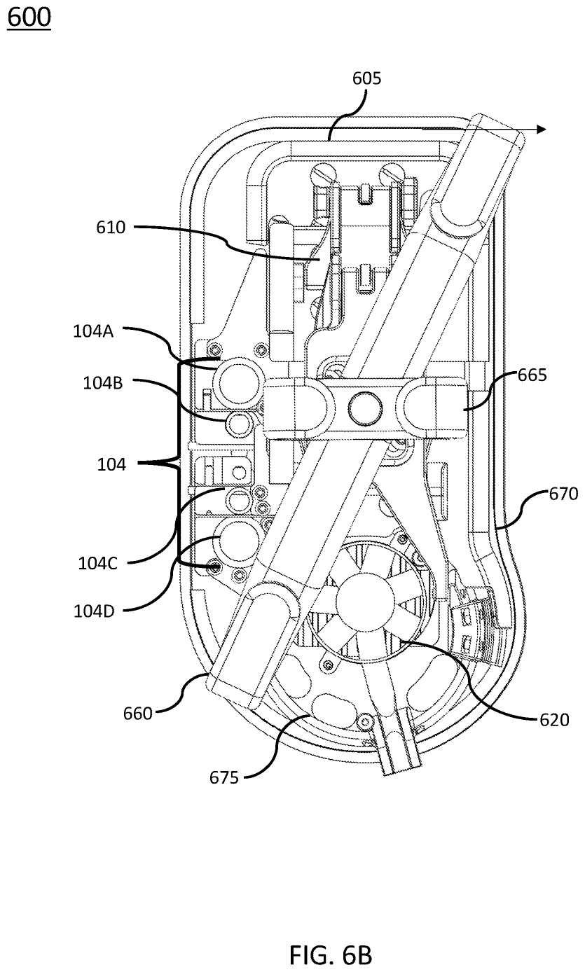

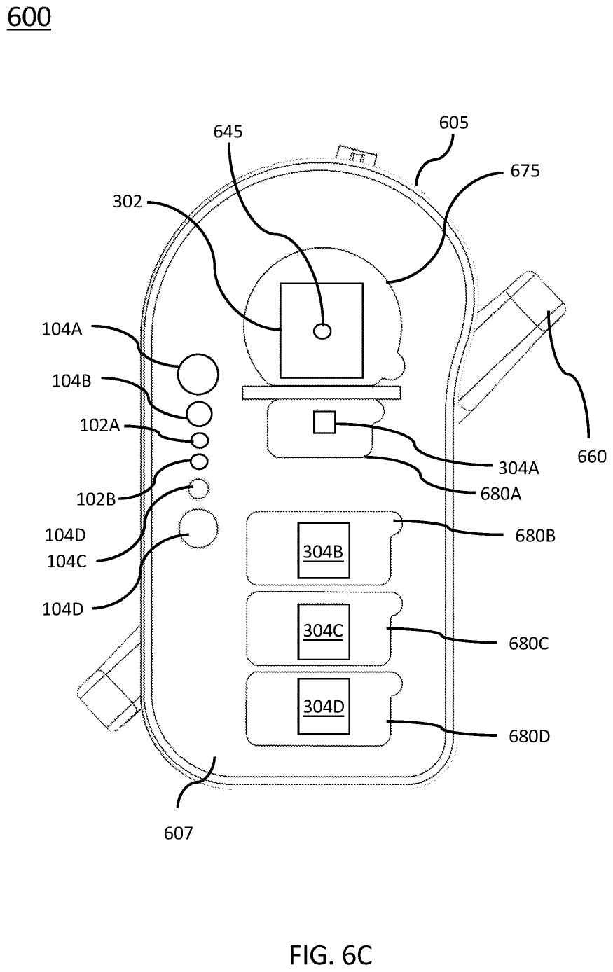

FIGS. 6A-6C provide illustrations of a third exemplary multi-function transabdominal fetal oximetry sensor 600 that includes components of frequency domain sensor system 101 and continuous wave sensor system 301 within the same housing 605. Third exemplary multi-function transabdominal fetal oximetry sensor 600 may be configured to cooperate with and/or receive instructions from a controller system like controller system 540 and/or a computer like computer 1310.

In particular, FIG. 6A provides a top view of a third exemplary multi-function transabdominal fetal oximetry sensor 600 with a swiveling handle 660 in a first orientation and FIG. 6B provides a top view of third exemplary multi-function transabdominal fetal oximetry sensor 600 with swiveling handle 660 in a second orientation after it has swiveled from the first orientation. Multi-function transabdominal fetal oximetry sensor 600 includes, among other things, an optional first arm 610, an optional second arm 615, a frequency domain detector array 104 including a first detector 104A, a second detector 104B, a third detector 104C, and a fourth detector 104D, swiveling handle 660, a stationary handle 665, a frequency domain light source 102, a continuous wave light source housing 620, a continuous wave detector housing 670, a continuous wave light source housing 675. In some embodiments, first detector 104A, second detector 104B, third detector 104C, and/or fourth detector 104D may be an opening, window, or other mechanism for optically coupling to a fiber optic or other optical cable that may receive an optical signal (e.g., an optical signal emanating from a pregnant mammal's abdomen) for forwarding on to a photodetector. Frequency domain light source 102 may be, for example, an LED and/or an opening, window, or other mechanism for coupling to a fiber optic or other optical cable that may supply an optical signal to a multi-function sensor.

FIG. 6C is a bottom view of multi-function transabdominal fetal oximetry sensor 600 and shows swiveling handle 660, a first and second light source 102A and 102B, and first, second, third, and fourth detectors 104A, 104B, 104C, and 104D, respectively of frequency domain array. In addition, the bottom view of FIG. 6C also shows a continuous wave detector array including first, second, third, and fourth detectors 304A, 304B, 304C, and 304D as well as windows 680, or coverings, for each respective detector. In particular, multi-function transabdominal fetal oximetry sensor 600 includes a first detector window 680A that covers first continuous wave detector 304A; a second detector window 680B that covers second continuous wave detector 304B; a third detector window 680C that covers third continuous wave detector 304C, and a fourth detector window 680D that covers fourth continuous wave detector 304D. Detector windows 680 may be configured to, for example, protect their respective detector, filter and/or polarize incoming optical signals and/or photons to, for example, remove ambient visible light and/or light of undesired wavelengths.

As may be seen in FIG. 6C, multi-function transabdominal fetal oximetry sensor 600 also includes a continuous wave light source 302 that may comprise a plurality of light sources 302 and include a central point 645 housed within continuous wave light source housing 675. In some embodiments, light sources 302 within continuous wave light source housing 675 may be positioned as closely as possible to one another so that light emitted from each individual LED is as close to center point 445 as possible, which may assist with a nearly homogenous output of light across a plurality of light sources included in continuous wave light source 302. In some cases, a displacement between each light source 302 within continuous wave light source housing 675 from center point 445 may be known and used in calculations corresponding to light emitted by each of the light sources 302. Light emitted by light sources 302 within continuous wave light source housing 675 may travel through a pregnant mammal's abdomen and, in some cases, be incident on the fetus therein, and then be emitted from the pregnant mammal's abdomen (via, for example, backscattering and/or reflection) and detected by first, second, third, and/or fourth detector(s) 304A, 304B, 304C, and 304D.

In some embodiments, a patient facing side of multi-function transabdominal fetal oximetry sensor 600 and/or components attached thereto may be configured to be flexible and, for example, be configured to curve in response to, for example, a curvature of a pregnant mammal's abdomen so that one or more components of multi-function transabdominal fetal oximetry sensor 600 (e.g., continuous wave light source housing 675, first-fourth continuous wave detectors 304A-304D, frequency domain light sources 102A and 102B, and/or first-fourth frequency domain detectors 104A-104D) may positioned proximate to and/or abut the pregnant mammal's abdomen regardless of a curvature thereof. This flexibility may be achieved via, for example, one or more hinges, flexible components (e.g., flexible housings for light sources and/or detectors and/or a flexible printed circuit board) and/or a flexible patient-facing surface 607 of housing 605.

Swiveling handle 660 of multi-function transabdominal fetal oximetry sensor 600 may be in the first orientation when, for example, in an open, or flat, position prior to attachment to a pregnant mammal's abdomen and, once in position (e.g., pressed against the pregnant mammal's abdomen) swiveling handle 660 may be turned, or rotated, into the second orientation as shown in FIG. 6B. Rotating swiveling handle 660 in this manner may activate (e.g., press downward) components of multi-function transabdominal fetal oximetry sensor 600 (arms etc.) so that a shape of the patient-facing side of multi-function transabdominal fetal oximetry sensor 600 conforms to a shape of the pregnant mammal's abdomen. Additionally, or alternatively, rotating swiveling handle 660 may facilitate cooperation with a strap (not shown) that is fed through an opening 830 in swiving handle 660 and wrapped around the pregnant mammal's abdomen to, for example, hold multi-function transabdominal fetal oximetry sensor 600 in place. In some embodiments, one or more components (e.g., swiveling handle 660) may be configured to cooperate with one or more straps that may wrap around the pregnant mammal's abdomen to hold multi-function transabdominal fetal oximetry sensor 600 in place.

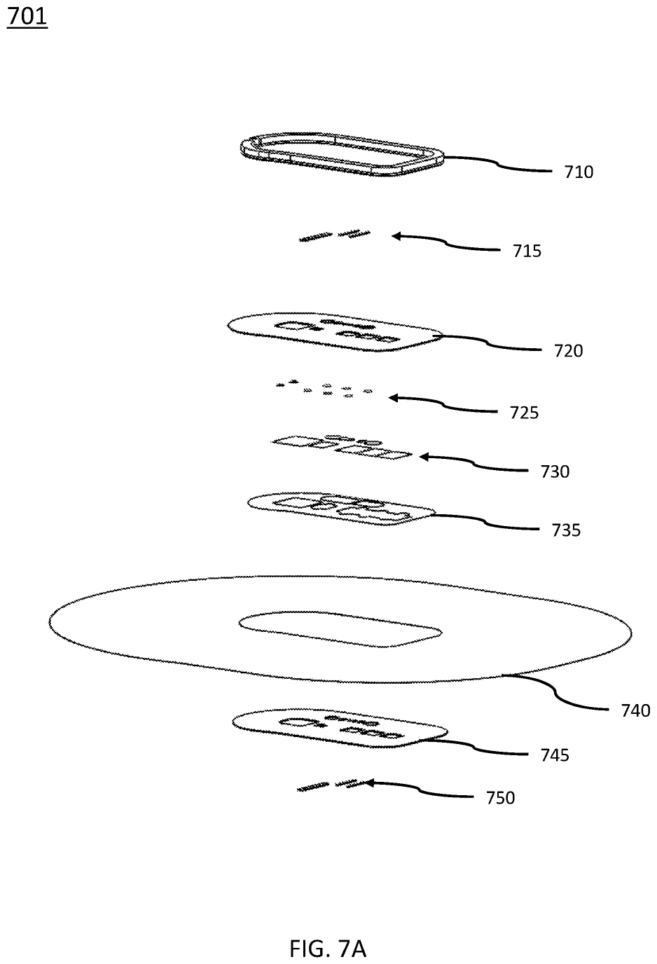

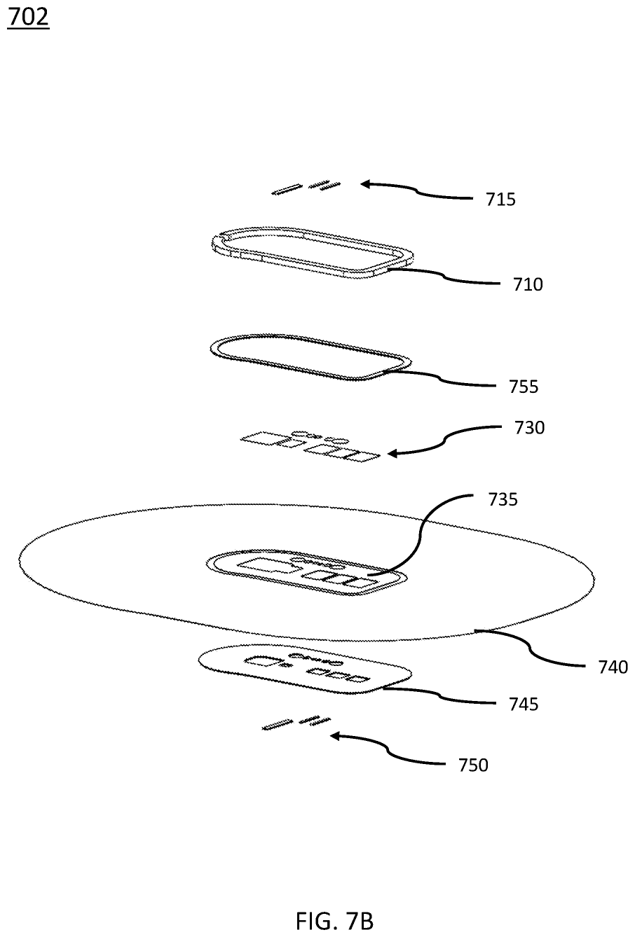

FIG. 7A is an exploded view of a first exemplary flexible patient interface assembly 701 configured for cooperation with multi-function transabdominal fetal oximetry sensor 600. Going from top to bottom, as oriented in FIG. 7A, flexible patient interface assembly 701 includes a ring 710 configured for acceptance of a patient-facing side of multi-function transabdominal fetal oximetry sensor 600 therein. Ring 710 may be configured to have a tight fit with multi-function transabdominal fetal oximetry sensor 600 so that, for example, ambient light and/or liquids cannot penetrate an interface between multi-function transabdominal fetal oximetry sensor 600 and ring 710 and/or penetrate an interface between multi-function transabdominal fetal oximetry sensor 600 and flexible patient interface assembly 701. Additionally, or alternatively, ring 710 may be configured to hold multi-function transabdominal fetal oximetry sensor 600 in place via, for example, friction and/or a mechanical device (e.g., clip or track) so that it may be correctly seated, and held in place, within ring 710. In some cases, ring 710 may be manufactured from foam, plastic, or another suitable material. Flexible patient interface assembly 701 may further include one or more light-blocking strips 715, which may be made from, for example, foam or rubber and may be configured and/or arranged to optically isolate one or more components (e.g., light sources and/or detectors) of multi-function transabdominal fetal oximetry sensor 600. Light-blocking strips 715 may be configured and/or arranged within first exemplary flexible patient interface assembly to prevent light from, for example, a frequency domain light source into continuous wave detector and vice versa. Flexible patient interface assembly 701 may further include a top sheet 720 that may be made from, for example, foam or rubber and may be configured and/or arranged to, for example, hold one or more components of multi-function transabdominal fetal oximetry sensor 600 in place and/or optically isolate one or more components of multi-function transabdominal fetal oximetry sensor 600. Optionally, flexible patient interface assembly 701 may include one or more magnets or ferromagnetic materials 725 configured to work in conjunction with corresponding elements of the multi-function transabdominal fetal oximetry sensor 600 to, for example, facilitate alignment and/or attachment of one or more components of flexible patient interface assembly 701.

In addition, flexible patient interface assembly 701 may include a set of windows 730 sized, arranged, and configured to cover continuous wave light source housing 675, first-fourth continuous wave detectors 304A, 304B, 304C, and/or 304D, frequency domain light source 102A and/or 102B, and/or first-fourth frequency domain detectors 104A, 104B, 104C, and/or 104D while allowing for the passage of light from frequency domain or continuous wave light sources 102 or 302 into a pregnant mammal's abdomen and from the pregnant mammal's abdomen into first, second, third, or fourth continuous wave detectors 304A, 304B, 304C, or 304D, or first, second, third, or fourth frequency domain detectors 104A, 104B, 104C, and/or 104D. In some embodiments, one or more of the windows included in set of windows 730 may provide for the unobscured passage of light and/or may be configured to filter light passing therethrough in order to, for example, filter out ambient light and/or amplify desired wavelengths of light. Windows of set of windows 730 may be positioned within a lateral support sheet 735 configured to, for example, hold the windows in place. Additionally, or alternatively, one or more of the windows included in set of windows 730 may be flexible.

Flexible patient interface assembly 701 may include an optical blanket 740 configured to optically isolate multi-function transabdominal fetal oximetry sensor 600 and/or a pregnant mammal's abdomen from ambient light. Optical blanket 740 may be positioned between lateral support sheet 735 and bottom sheet 745 and may be made from any opaque material including, but not limited to, fabric, plastic, and foam. In some embodiments, optical blanket 740 may be configured to be affixed to a pregnant mammal's abdomen via, for example, an adhesive positioned on a patient-facing side of optical blanket 740. Flexible patient interface assembly 701 may also include a bottom sheet 745 that may be configured to align components of flexible patient interface assembly 701 and hold them in place. Optionally, flexible patient interface assembly 701 may also have one or more light-blocking strips 750 configured and arranged to optically isolate one or more components of multi-function transabdominal fetal oximetry sensor 600. A lower surface of flexible patient interface assembly 701 may be configured to be resistant to water and other chemicals and/or solutions (e.g., sweat, ultrasound gel and/or optically conducing gel) that may be placed upon and/or secret from the pregnant mammal's abdomen so that, for example, multi-function transabdominal fetal oximetry sensor 600 is not contaminated with and/or destroyed by such substances.

FIG. 7B is an exploded view of a second exemplary flexible patient interface assembly 702, which is similar to first exemplary flexible patient interface assembly 701 except that second exemplary flexible patient interface assembly 702 does not include top sheet 720 or magnets 725. In some embodiments, one or more components of first and/or second flexible patient interface assembly 701 or 702 may be configured to be flexible but not stretchy. In this way, a relatively distance between components may be held constant.

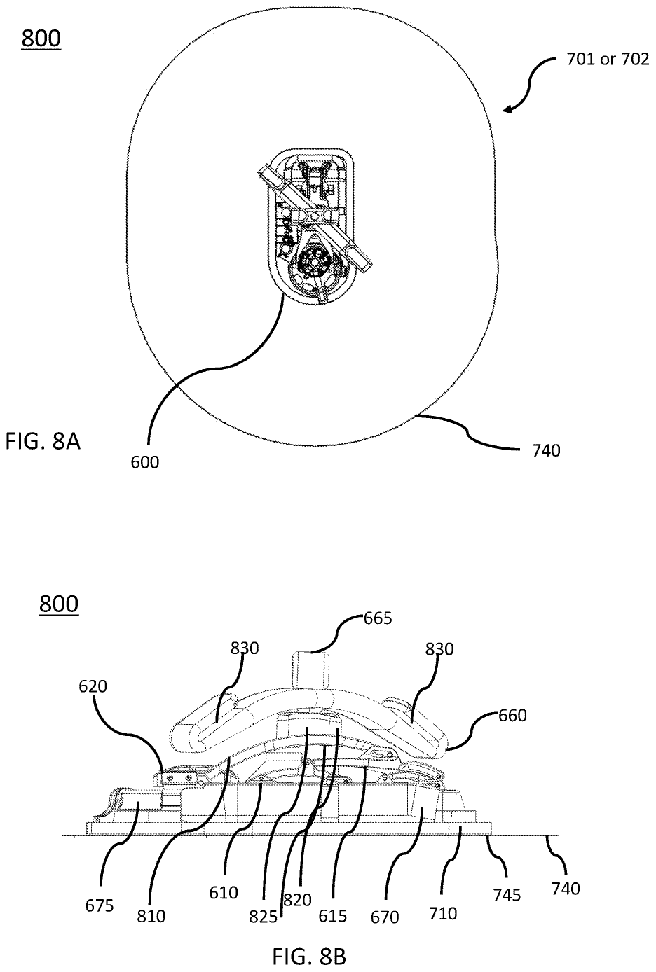

FIG. 8A is a top view and FIG. 8B is a side view of a system 800 including multi-function transabdominal fetal oximetry sensor 600 seated within ring 710 of first or second exemplary flexible patient interface assembly 701 or 702. Swiveling handle 660 of multi-function transabdominal fetal oximetry sensor 600 may be configured as a handle that enables a user to place multi-function transabdominal fetal oximetry sensor 600 at a desired location on, for example, an abdomen of a pregnant mammal so that one or more measurements may be taken. Swiveling handle 660 may be configured to swivel, or rotate, between first (shown in FIG. 6A) and second (shown in FIG. 6B) orientations via a swiveling mechanism, or shaft, 825 positioned within a rotational component housing 820. The swiveling of swiveling handle 660 may be assisted by stationary handle 665, which may be configured to maintain its position and/or orientation while swiveling handle 660 is being rotated between the first and second orientations. In some embodiments, rotating swiveling handle 660 between the first and second orientations may initiate the application of force to an arm 810. Additionally, or alternatively, a normal downward (as oriented in the figure) force applied to swiveling handle 660 may cause arms 810, 610, and/or 615 to move, or pivot, thereby a lower surface of multi-function transabdominal fetal oximetry sensor 600 and/or first or second flexible patient interface assembly 701 or 702. The force applied to arm 810 may be transferred to first and/or second arm 610 and 615 and this force may be transferred to first or second flexible patient interface assembly 701, 702, or portions thereof so that flexible patient interface assembly 701 or 702 curves in order to, for example, match, or form fit to, a curved portion of the pregnant mammal's abdomen.

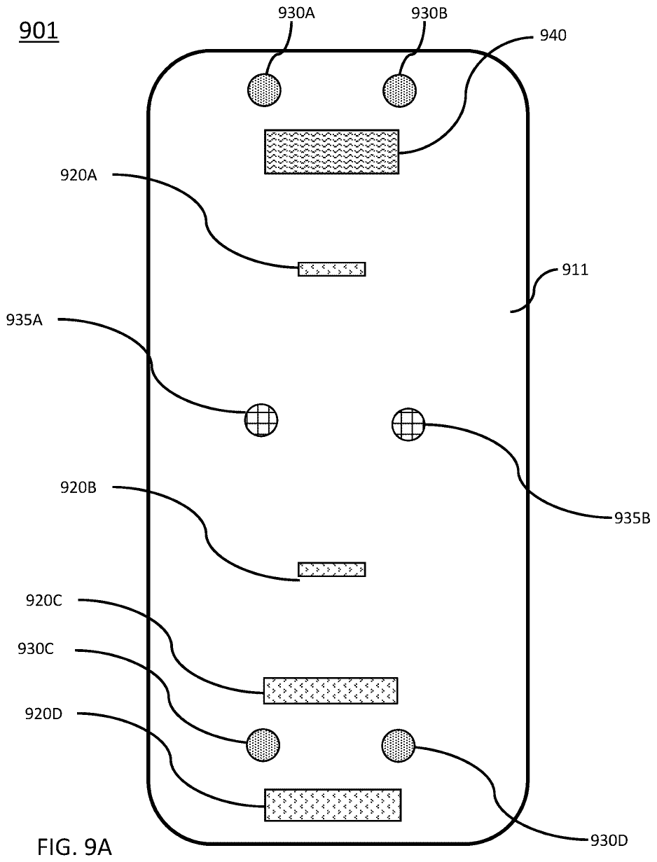

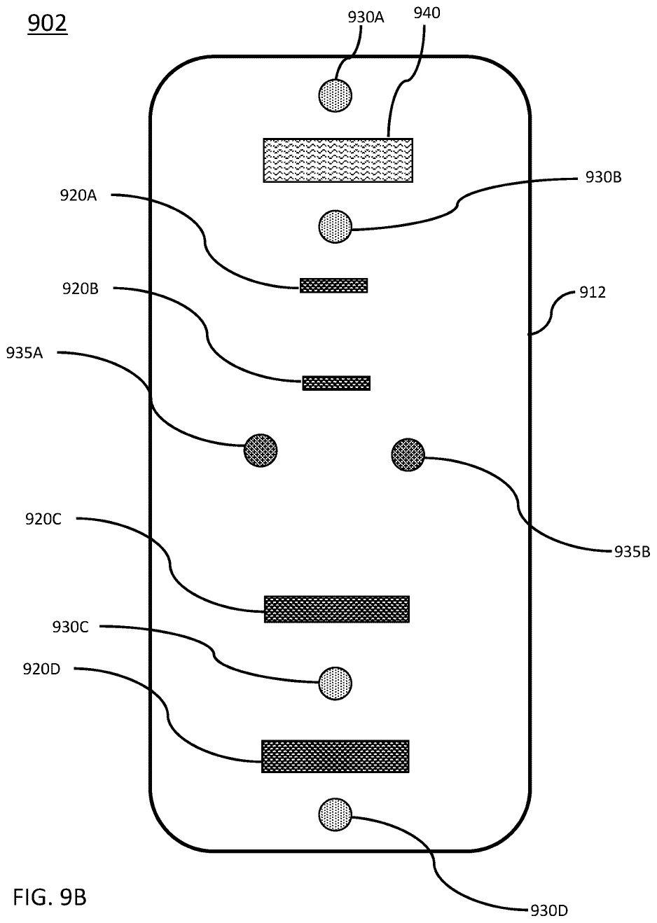

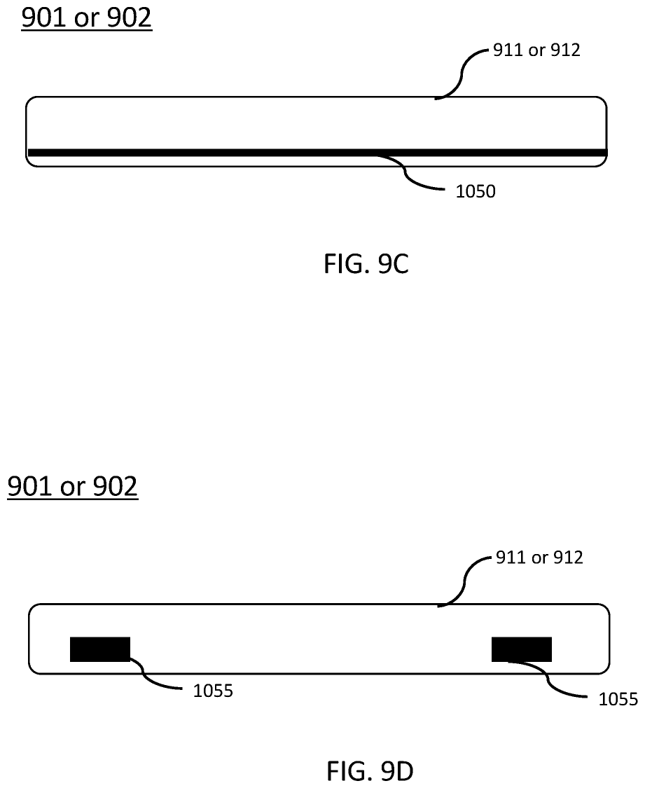

FIGS. 9A and 9B provide bottom views of a fourth and a fifth exemplary multi-function transabdominal fetal oximetry sensor 901 and 902, respectively. Fourth exemplary multi-function transabdominal fetal oximetry sensor 901 may be configured to communicate with, cooperate with, and/or receive instructions from a controller system like controller system 540 and/or a computer like computer 1310 as, for example, disclosed herein.

Fourth exemplary multi-function transabdominal fetal oximetry sensor 901 includes a housing 911 that houses a frequency domain system that may be similar to frequency domain sensor system 101. In particular, the frequency domain system of multi-function transabdominal fetal oximetry sensor 901 includes a first frequency domain light source 930A, a second frequency domain light source 930B, a third frequency domain light source 930C, and a fourth frequency domain light source 930D that are configured to emit an optical signal that may be projected into an abdomen of a pregnant mammal. First and second frequency domain light sources 930A and 930B are positioned in a row proximate to a first, or top (as oriented in FIG. 9A) edge and third and fifth frequency domain light sources 930C and 930D are positioned in a row proximate to a second, or bottom, edge of fourth exemplary multi-function transabdominal fetal oximetry sensor 901. First, second, third, and/or fourth frequency domain light sources 930A, 930B, 930C, and/or 930D may be similar to frequency domain light source(s) 102.

The frequency domain system of fourth exemplary multi-function transabdominal fetal oximetry sensor 901 further comprises a first frequency domain detector 935A and a second frequency domain detector 935B positioned in a row at an approximate vertical center of fourth exemplary multi-function transabdominal fetal oximetry sensor 901 so that the row of first frequency domain detector 935A and second frequency domain detector 935B is positioned in a center of fourth exemplary multi-function transabdominal fetal oximetry sensor 901 approximately equidistant between the row of first and second frequency domain light sources 930A and 930B and the row of third and fourth frequency domain light sources 930C and 930D.

First and second frequency domain detectors 935A and 935B may be configured to detect a portion of the optical signal emitted by one or more of first, second, third, and/or fourth frequency domain light source(s) 930A, 930B, 930C, and/or 930D, respectively, that has been reflected and/or backscattered from the pregnant mammal's abdomen. Fourth exemplary multi-function transabdominal fetal oximetry sensor 901 further includes a continuous wave sensor system that includes an optical source 940 and a plurality of a first, second, third, and fourth continuous wave detectors 920A, 920B, 920C, and 920D, respectively, positioned at various distances from optical source 940. Optical source 940 may be, for example, a plurality of LEDs and/or laser inputs (e.g., fiber optic cables coupled to a laser source). In some cases, optical source 940 may be similar to continuous wave light sources 302.

The components of fifth exemplary multi-function transabdominal fetal oximetry sensor 902 of FIG. 9B are similar to those in the fourth exemplary multi-function transabdominal fetal oximetry sensor 901 but are arranged in a different configuration within a housing 912 on the patient-facing side of fifth exemplary multi-function transabdominal fetal oximetry sensor 902 as shown. Fifth exemplary multi-function transabdominal fetal oximetry sensor 902 may be configured to communicate with, cooperate with, and/or receive instructions from a controller system like controller system 540 and/or a computer like computer 1310 as, for example, disclosed herein.

In some embodiments, the arrangement of components of first, second, third, fourth, and/or fifth exemplary multi-function transabdominal fetal oximetry sensors 501, 502, 600, 901, and/or 902, may be configured so that maternal and/or fetal anatomy and/or tissues may be probed longitudinally (i.e., depth) and/or laterally (i.e., width) with the arrangement of first frequency source 930A, second frequency source 930B, third frequency source 930C, and fourth frequency source 930D of fifth exemplary multi-function transabdominal fetal oximetry sensor 902 providing more longitudinal information regarding maternal, or frequency-domain detected, geometry than, for example, the arrangement of frequency sources in fourth exemplary multi-function transabdominal fetal oximetry sensors 901. A potential advantage of fifth multi-function transabdominal fetal oximetry sensor 902 when compared with fourth multi-function transabdominal fetal oximetry sensor 901, is that fifth multi-function transabdominal fetal oximetry sensor 902 may allow for computation of a difference and/or slope across two relatively long source-detector distances, which may potentially increase a sensitivity to optical changes (e.g., changes in maternal and/or fetal optical properties and/or position) at greater depths within the maternal abdomen.

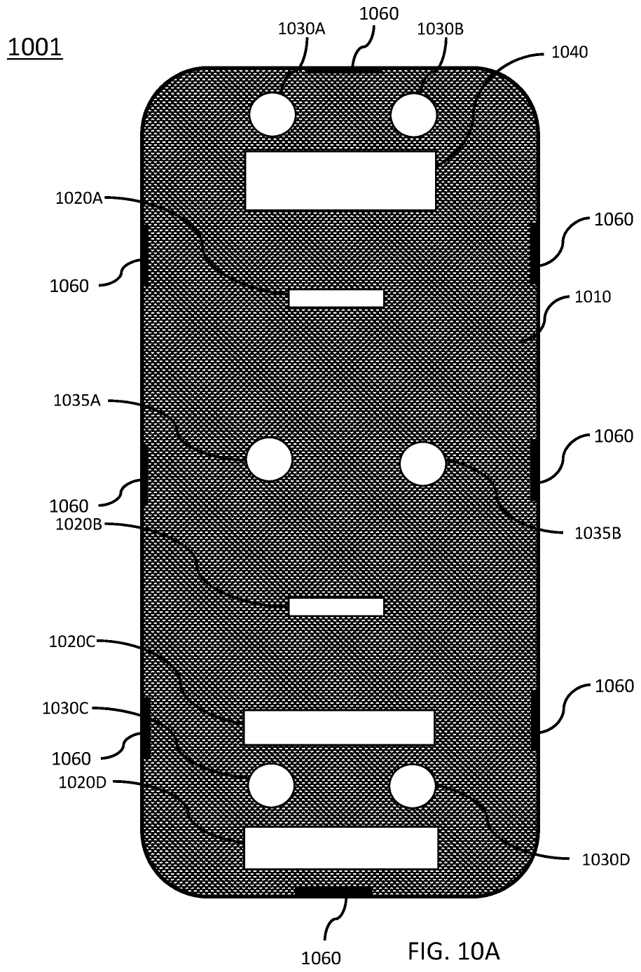

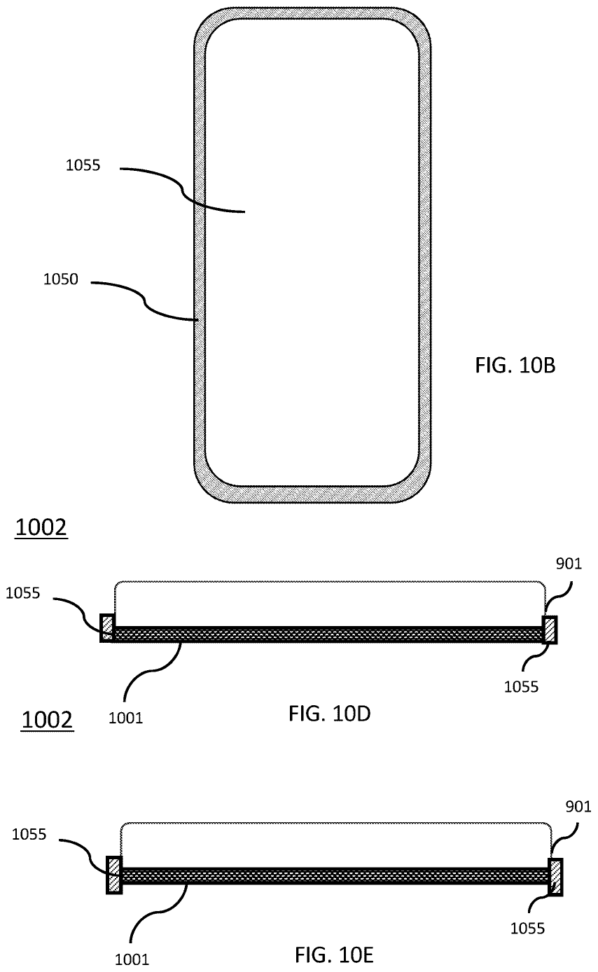

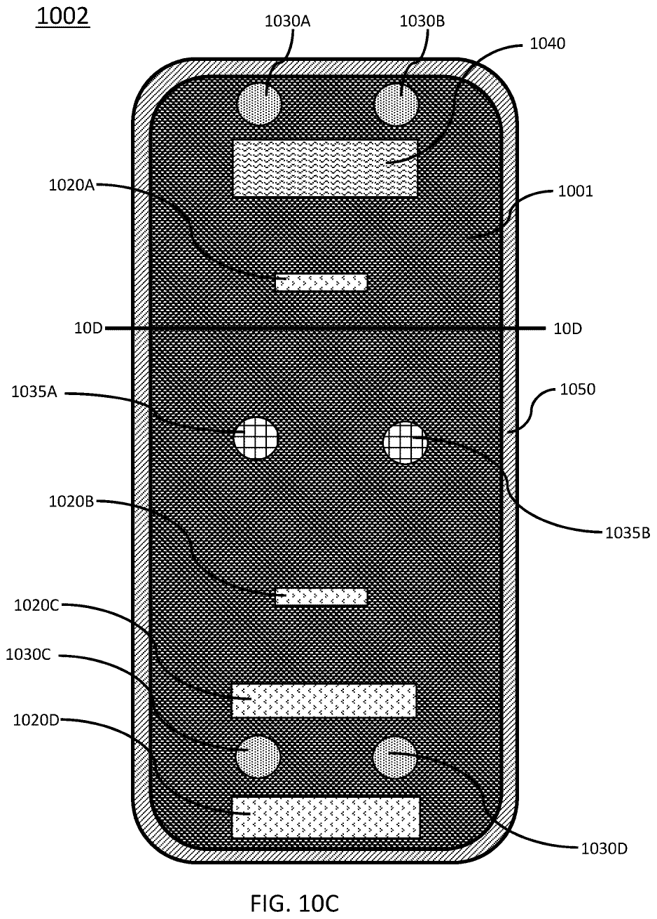

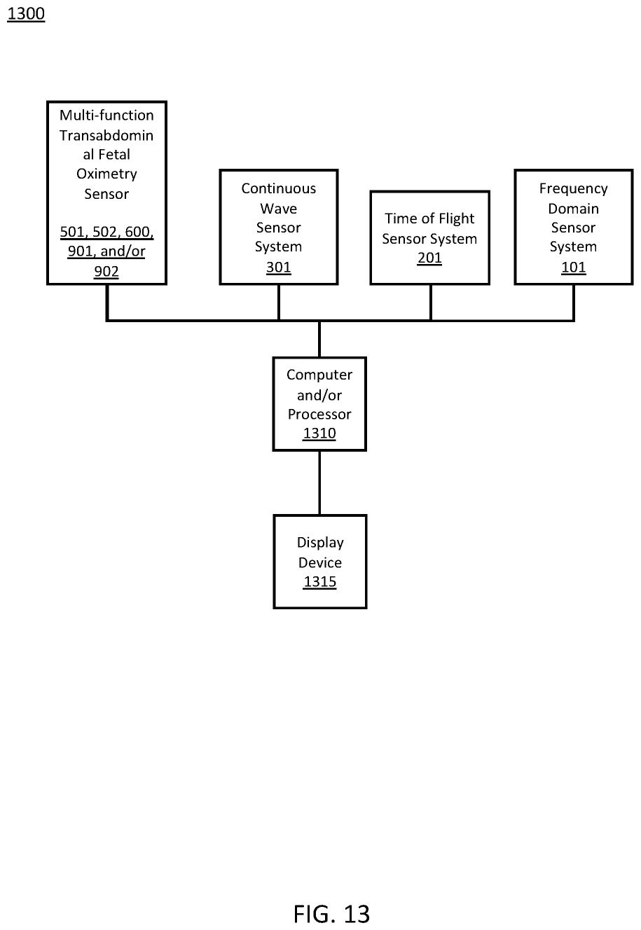

FIG. 9C is a side view of multi-function transabdominal fetal oximetry sensor 901 or 902 in which a track 1050 extends along a lower (as oriented in the figure) edge of housing 911 or 1012. Track 1050 may be configured to cooperate with one or more attachment mechanisms provided by, for example, a patient interface such as patient interface 1001 or 1100 as shown in FIGS. 10A and 11A, respectively, and discussed below and/or an optical blanket like optical blanket 1301 as shown in FIG. 13A and discussed below. Track 1050 may extend around all four sides of multi-function transabdominal fetal oximetry sensor 901 or 902 and/or a portion thereof. For example, track 1050 may extend along a portion of the sides of multi-function transabdominal fetal oximetry sensor 901 or 902 that correspond to the one or more attachment mechanisms of a patient interface and/or optical blanket. Additionally, or alternatively, multi-function transabdominal fetal oximetry sensor 901 or 902 may include one or more mechanisms 1055 (e.g., a clamp, snap, or clip) configured to attach multi-function transabdominal fetal oximetry sensor 901 or 902 to a patient interface and/or optical blanket as shown in FIG. 9D. Attachment mechanisms 1055 may be positioned on one or more sides of multi-function transabdominal fetal oximetry sensor 901 or 902. Multi-function transabdominal fetal oximetry sensor may also include a track like track 1050 and/or an attachment mechanism like attachment mechanisms 1055 that may be configured to cooperate with a patient interface and/or optical blanket such as the patient interfaces and optical blankets disclosed herein.

FIGS. 10A-10D provide schematic diagrams of patient interfaces configured to cooperate with multi-function transabdominal fetal oximetry sensor 901 to, for example, assist with attaching multi-function transabdominal fetal oximetry sensor 901 to a pregnant mammal's abdomen and/or prevent optical shunting between components of multi-function transabdominal fetal oximetry sensor 901 in a manner similar to, for example, patient interface 401 and/or 402.

FIG. 10A is a block diagram of an exemplary patient interface 1001 for use with multi-function transabdominal fetal oximetry sensor 901. Patient interface 1001 may be a sheet of material 1010 (e.g., foam, fabric, plastic, etc.) sized, shaped, and configured to cover the patient-facing surface of multi-function transabdominal fetal oximetry sensor 901 without obscuring a source or detector thereof. In some embodiments, a thickness (e.g., 0.5-10 mm) of patient interface 1001/sheet of material 1010 may be known so that it may be factored into, for example, geometrical and/or temporal calculations of light exiting and/or entering multi-function transabdominal fetal oximetry sensor 901. Patient interface 1001 may include a plurality of windows sized, shaped, and arranged within patient interface 1001 to align with the light sources and/or detectors of multi-function transabdominal fetal oximetry sensor 901. For example, material 1010 may include a first, second, third, and fourth frequency domain light source window 1030A, 1030B, 1030C, and 1030D that are respectively sized, positioned, and arranged within sheet of material 1010 to correspond to a size, position, and arrangement of first, second, third, and fourth frequency domain light sources 930A, 930B, 930C, and 930D, respectively. Material 1010 also includes a first and second frequency domain detector windows 1035A and 1035B that are sized, positioned, and arranged, to correspond to a size, position, and arrangement of first and second frequency domain detectors 935A and 935B, respectively. In addition, material 1010 includes a continuous wave light source window 1040 sized, positioned, and arranged, to correspond to a size, position, and arrangement of continuous wave light source 940. Material 1010 may further include a first, second, third, and fourth continuous wave detector window 1020A, 1020B, 1020C, and 1020D, respectively, that are sized, positioned, and arranged, to correspond to a size, position, and arrangement of first, second, third, and fourth continuous wave detectors 920A, 920B, 920C, and 920D, respectively. Optionally, patient interface 1001 also includes a plurality (in this case, eight) attachment mechanisms 1060 configured to attach to multi-function transabdominal fetal oximetry sensor 901 via, for example, track 1050 and/or attachment mechanisms 1055.

FIG. 10B is a top view of a ring 1050 with a center opening 1155 configured to accept insertion of frequency domain sensor system 101, time-of-flight sensor system 201, continuous wave sensor system 301, and/or multi-function transabdominal fetal oximetry sensor 501, 502, 901, and/or 902 therein without obscuring a component thereof. Ring 1050 may be similar in form, function, and/or operation to ring 440 and may be configured to and cover a junction between frequency domain sensor system 101, time-of-flight sensor system 201, continuous wave sensor system 301, and/or multi-function transabdominal fetal oximetry sensor 501, 502, 901, and/or 902 and the pregnant mammal's abdomen and/or patient interface 1001 as shown in, for example, FIGS. 10C-10E, wherein FIG. 10C provides a top view of an assembly 1002 of multi-function transabdominal fetal oximetry sensor 901, patient interface 1001, and ring 1155 and shows how each of the windows of patient interface 1001 aligns with its respective component so that it is not obscured. FIG. 10D provides a cross-section view of assembly 1002 along line 10D shown in FIG. 10C. The cross-section of FIG. 10D shows how ring 1155 covers an interface between patient interface 1001 and multi-function transabdominal fetal oximetry sensor 901 so that light cannot enter this interface. In some embodiments ring 1155 may extend down below the patient-facing side of patient interface 1001 as shown in the cross-section of FIG. 10E (also taken along bisecting line 10D) and, in this embodiment, ring 1155 may be configured to conform to the pregnant mammal's anatomy to, for example, form a tight optical coupling between patient interface 1001/multi-function transabdominal fetal oximetry sensor 901 and the pregnant mammal's abdomen.

FIG. 11A is a block diagram of an exemplary patient interface 1100 for use with multi-function transabdominal fetal oximetry sensor 902. Patient interface 1100 may be similar to patient interface 1001 except that the windows present therein correspond to positions, sizes, and locations of the light sources and detectors of multi-function transabdominal fetal oximetry sensor 902. For example, material 1112 may include a first, second, third, and fourth frequency domain light source window 1130A, 1130B, 1130C, and 1130D sized, positioned, and arranged, to correspond to a size, position, and arrangement of first, second, third, and fourth frequency domain light sources 930A, 930B, 930C, and 930D, respectively. Material 1110 also includes a first and second frequency domain detector windows 1135A and 1135B that are sized, positioned, and arranged to correspond to a size, position, and arrangement of first and second frequency domain detectors 935A and 935B, respectively. In addition, material 1110 includes a continuous wave light source window 1140 sized, positioned, and arranged, to correspond to a size, position, and arrangement of continuous wave light source 940. Material 1110 may further include a first, second, third, and fourth continuous wave detector window 1120A, 1120B, 1120C, and 1120D, respectively, that are sized, positioned, and arranged to correspond to a size, position, and arrangement of first, second, third, and fourth continuous wave detectors 920A, 920B, 920C, and 920D, respectively. Optionally, patient interface 1100 also includes a plurality (in this case, eight) attachment mechanisms 1160 configured to attach to multi-function transabdominal fetal oximetry sensor 901 via, for example, track 1050 and/or attachment mechanisms 1055.

FIG. 11B is a diagram of an assembly 1101 of patient interface 1100 and ring 1155 that may be similar to assembly 1002 with the exception that assembly 1101 includes patient interface 1100 instead of patient interface 1001. In some embodiments, patient interface 1001 and/or 1100 may not include attachment mechanisms 1060 or 1160, respectively. For example, when patient interface 1001 and/or 1100 includes an adhesive on the multi-function transabdominal fetal oximetry sensor-facing surface, frequency domain sensor system 101, time-of-flight sensor system 201, continuous wave sensor system 301, and/or multi-function transabdominal fetal oximetry sensor 501, 502, 901, and/or 902 may be coupled to the patient interface without the need for attachment mechanisms 1060 or 1160. Additionally, or alternatively, when patient interface 1001 and/or 1100 are included in an assembly including ring 1155, frequency domain sensor system 101, time-of-flight sensor system 201, continuous wave sensor system 301, and/or multi-function transabdominal fetal oximetry sensor 501, 502, 901, and/or 902 may fit within ring 1155 and may be held therein as a result of, for example, friction and/or compressive forces exerted by ring 1155 on the external surface of the frequency domain sensor system 101, time-of-flight sensor system 201, continuous wave sensor system 301, and/or multi-function transabdominal fetal oximetry sensor 501, 502, 901, and/or 902.

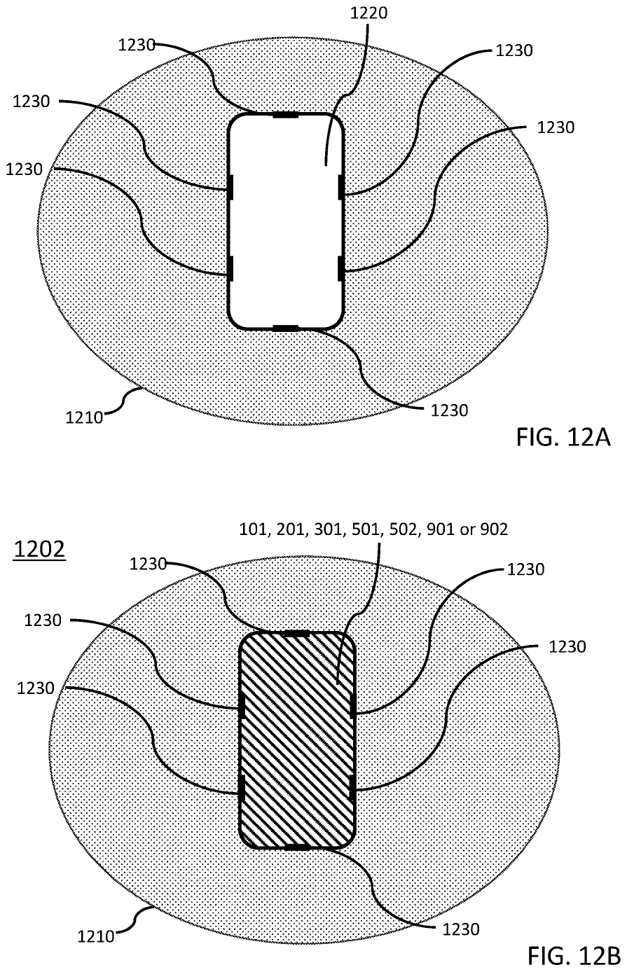

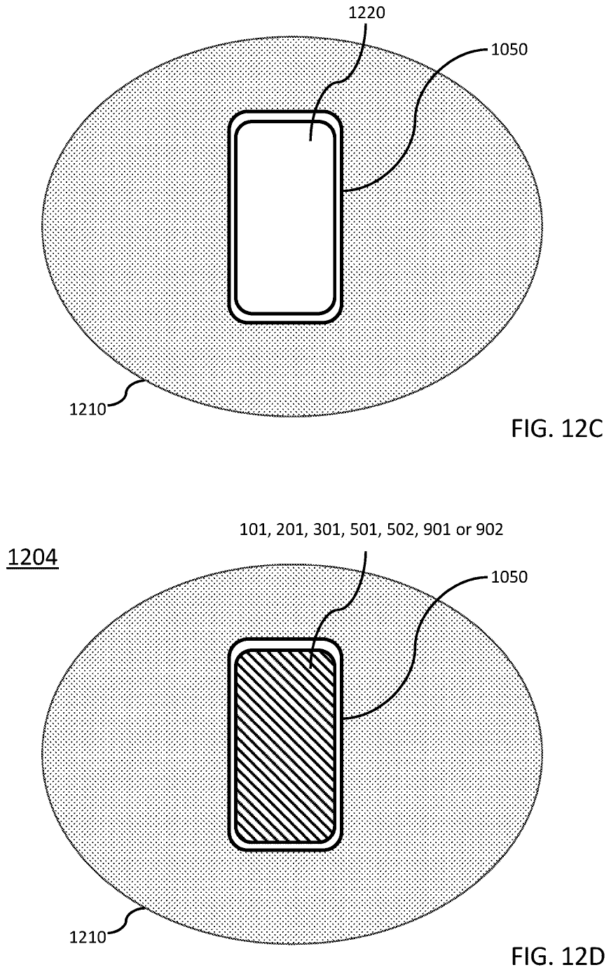

FIG. 12A is a diagram of an exemplary optical blanket 1210 that includes an opening 1220 sized, shaped, and/or configured to accept insertion of, for example, frequency domain sensor system 101, time-of-flight sensor system 201, continuous wave sensor system 301, and/or multi-function transabdominal fetal oximetry sensor 501, 502, 901, and/or 902 therein without obscuring one or more components thereof. Optical blanket 1210 may be configured to assist in the formation of a tight optical coupling between frequency domain sensor system 101, time-of-flight sensor system 201, continuous wave sensor system 301, and/or multi-function transabdominal fetal oximetry sensor 501, 502, 901, and/or 902 and the pregnant mammal's abdomen and/or prevent ambient light from entering the pregnant mammal's abdomen. Optical blanket 1210 made from any opaque material (e.g., fabric, foam, plastic, etc.) that is capable of covering and conforming to a portion of a pregnant mammal's abdomen and block entry of ambient light therein. Optical blanket 1210 may be held in place on the pregnant mammal's abdomen via, for example, gravity, an adhesive, and/or compression via, for example, a strap or other device that holds optical blanket 1210 in place over the pregnant mammal's abdomen. In some embodiments, placement of optical blanket 1210 by a clinician may be responsive to fetal position (e.g., placed so that opening 1220 corresponds to a preferred region of the fetus from which to take a measurement, such as the head or back) as determined via, for example, clinical observations of an ultrasound or other image of the fetus or pregnant mammal's abdomen. Optionally, optical blanket 1210 may include a plurality of attachment mechanisms 1230 configured to engage with and/or couple to frequency domain sensor system 101, time-of-flight sensor system 201, continuous wave sensor system 301, and/or multi-function transabdominal fetal oximetry sensor 501, 502, 901, and/or 902 and/or a track (e.g., track 330 or 1050) or attachment mechanism (e.g., attachment mechanism 340 or 1055) thereof.

In some embodiments, a patient interface (e.g., patient interface 401, 402, 1001, 1002, 1100 or 1101) may be placed on the maternal abdomen prior to placement of optical blanket 1210. In these embodiments, the patient interface may be placed on and/or affixed to the pregnant mammal's abdomen responsively to fetal position and optical blanket 1210 may be placed on the pregnant mammal's abdomen by aligning the patient interface and opening 1220 so that the patient interface fits within opening 1220 and optical blanket 1210 surrounds the personal interface

Whether a patient interface has been placed on the pregnant mammal's abdomen, or not, frequency domain sensor system 101, time-of-flight sensor system 201, continuous wave sensor system 301, and/or multi-function transabdominal fetal oximetry sensor 501, 502, 901, and/or 902 may be placed within opening 1220 in an assembly 1202 as shown FIG. 12B and optionally attached thereto via attachment mechanisms 1230. In this way, positioning of one or more of frequency domain sensor system 101, time-of-flight sensor system 201, continuous wave sensor system 301, and/or multi-function transabdominal fetal oximetry sensor 501, 502, 901, and/or 902 on the pregnant mammal's abdomen may be repeatable (i.e., if removed, frequency domain sensor system 101, time-of-flight sensor system 201, continuous wave sensor system 301, and/or multi-function transabdominal fetal oximetry sensor 501, 502, 901, and/or 902 may be re-affixed to the same position on the pregnant mammal's abdomen using opening 1220 and/or a patient interface as a guide).

FIG. 12C is a diagram of an embodiment wherein opening 1220 is surrounded by ring 1050 that, on some occasions, may extend outward from optical blanket 1210 in the Z direction away from a patient-facing side of optical blanket 1210. Whether a patient interface has been placed on the pregnant mammal's abdomen, or not, frequency domain sensor system 101, time-of-flight sensor system 201, continuous wave sensor system 301, and/or multi-function transabdominal fetal oximetry sensor 501, 502, 901, and/or 902 may be placed within ring 1050 and opening 1220 in an assembly 1204 as shown FIG. 12D.

FIG. 13 provides a block diagram of an exemplary system 1300 for executing one or more methods disclosed herein. System 1300 includes a computer and/or processor 1310 configured to communicate with one or more of frequency domain sensor system 101, time-of-flight sensor system 201, continuous wave sensor system 301, and/or multi-function transabdominal fetal oximetry sensor 501, 502, 600, 901, or 902 and receive one or more signals therefrom. Computer and/or processor 1310 may include a memory programmed to store one or more sets of instructions for executing one or more processes described herein. Computer and/or processor 1310 may also be configured to process the received signals according to one or more processes described herein to, for example, characterize maternal and/or fetal tissue and/or determine fetal oximetry information and provide the fetal oximetry information to a display device (e.g., touch screen, display screen, computer monitor, etc.) for display to a user. In some embodiments, the computer and/or processor 1310 and/or display device may be resident within a housing for frequency domain sensor system 101, time-of-flight sensor system 201, continuous wave sensor system 301, and/or multi-function transabdominal fetal oximetry sensor 501, 502, 600, 901, and/or 902.

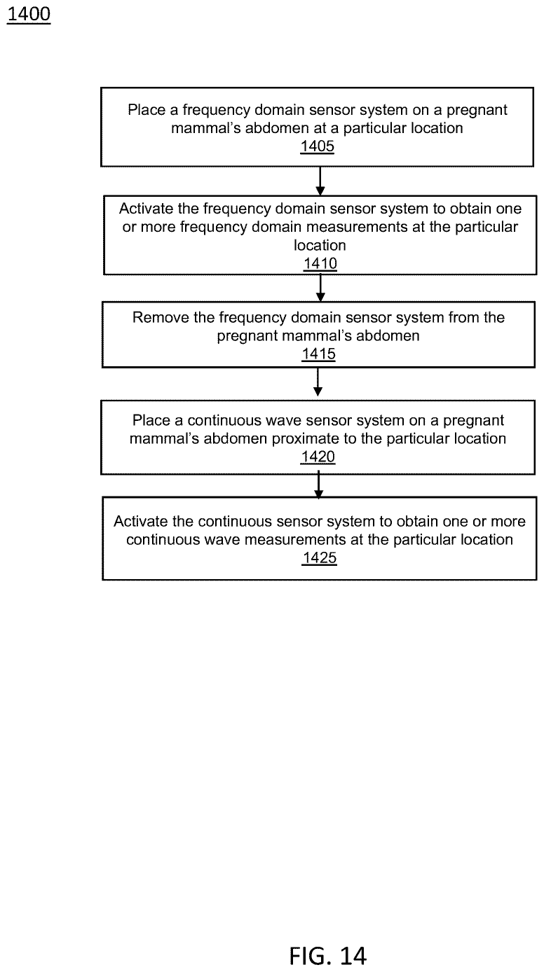

FIG. 14 provides a flowchart illustrating a process 1400 for using a frequency domain sensor system like frequency domain sensor system 101 (or components thereof) and a continuous wave sensor system like continuous wave sensor system 301 (or components thereof) to obtain frequency domain and continuous wave optical measurements of a pregnant mammal's abdomen and a fetus contained within the pregnant mammal's abdomen. Process 1400 may be executed by, for example, a user such as a clinician or the pregnant mammal using one or more systems and/or devices disclosed herein.

Initially, in step 1405, a frequency domain sensor system, such as frequency domain sensor system 101, may be placed on a pregnant mammal's abdomen in a particular location. On some occasions, fetal position and/or anatomy used to determine a frequency domain sensor system placement position may be determined via, for example, examination of an ultrasound image and/or using a Doppler ultrasound machine. In some embodiments, a patient interface (e.g., patient interface 401 or 402) and/or optical blanket (e.g., optical blanket 1210) compatible with the frequency domain and/or continuous wave sensor systems may be placed and/or affixed to the pregnant mammal's abdomen prior to execution of step 1405. In these embodiments, the patient interface and/or optical blanket may guide placement of the frequency domain sensor system and/or may assist in affixing the frequency domain system to the pregnant mammal's abdomen. Additionally, or alternatively, a patient-facing side of the frequency domain sensor system may be covered with a patient interface that, in some cases, may be removable.

In some embodiments, a test of an optical coupling between frequency domain sensor system and the pregnant mammal's abdomen may be performed prior to execution of step 1410 to determine whether or not an optical shunting between the frequency domain light sources and frequency domain detectors has occurred and, if so, a position and/or optical coupling of frequency domain sensor system may be adjusted to reduce or eliminate the optical shunting. If optical shunting is found to be present, a position, orientation, and/or setting of the frequency domain sensor system, patient interface, and/or optical blanket may be adjusted and/or a coupling between the frequency domain sensor system and patient interface, and/or optical blanket may be adjusted. Additionally, or alternatively, a degree of optical shunting may be determined and then applied to signal(s) received from the frequency domain sensor system to correct for the optical shunting. Once in position, in step 1410, the frequency domain sensor system may be activated (e.g., turned on or run through a routine) via, for example, the user to obtain one or more frequency domain measurements of the pregnant mammal's abdomen.

In step 1415, the frequency domain sensor system may be removed from the pregnant mammal's abdomen when, for example, one or more frequency domain measurements have been taken and a continuous wave sensor system like continuous wave sensor system 301 may then be placed on the pregnant mammal's abdomen at the same position/location from which the frequency domain sensor system has been removed (step 1420) so that one or more continuous wave measurements may be taken of the pregnant mammal's abdomen and fetus therein when the continuous wave sensor system is activated (step 1425). For embodiments where a patient interface and/or optical blankets are being used, step 1415 may be executed by removing the frequency domain sensor system from the patient interface and/or optical blanket and then step 1420 may be executed by attaching the continuous wave sensor system to the patient interface and/or optical blanket while maintaining an original position (i.e., a position prior to execution of step 1405) of the patient interface and/or optical blanket. In this way, the patient interface and/or optical blanket may assist with ensuring that a position of the continuous wave sensor system on the pregnant mammal's abdomen corresponds, or approximately corresponds, to a former position of the frequency domain sensor system. Additionally, or alternatively, a patient-facing side of the continuous wave sensor system may be covered with a patient interface that, in some cases, may be removable.

In some embodiments, a test of an optical coupling between continuous wave sensor system and the pregnant mammal's abdomen may be performed prior to execution of step 1425 to determine whether or not an optical shunting between the continuous wave light sources and continuous wave detectors has occurred and, if so, a position and/or optical coupling of continuous wave sensor system may be adjusted to reduce or eliminate the optical shunting. Additionally, or alternatively, a degree of optical shunting may be determined and then applied to signal(s) received from the continuous wave sensor system to correct for the optical shunting.

As noted above, in some embodiments, a patient interface such as patient interface 401 and/or an optical blanket such as optical blanket 1210 may be used during execution of process 1400. In some instances, the patient interface may be affixed to the frequency domain system and/or pregnant mammal's abdomen prior to execution of step 1405 and the patient interface may be configured to cooperate with the frequency domain sensor system via, for example, affixing to the frequency domain sensor system and/or having one or more windows that correspond to a frequency domain light source (frequency domain light source 102) and/or frequency domain detector (e.g., frequency domain detector 104). Frequency domain sensor system may be affixed to the patient interface via, for example, an adhesive, a ring like ring 440, an attachment mechanism like attachment mechanisms 340, and/or a track like track 330. Then, when frequency domain sensor system is removed from the pregnant mammal's abdomen (step 1415), the frequency domain sensor system may be disengaged from the patient interface via, for example, peeling it off an adhesive of the patient interface, disengagement of an attachment mechanism affixing the frequency domain sensor system to the patient interface, sliding the frequency domain sensor system out of a track of the patient interface, and/or lifting the frequency domain sensor system from the ring. Once the frequency domain sensor system is removed from the patient interface in step 1415, step 1420 may be executed by affixing the continuous wave sensor system to the patient interface via, for example, an adhesive, the ring, the attachment mechanism, and/or the track. Additionally, or alternatively, a patient interface may be attached to the continuous wave sensor system. In embodiments where a patient interface is not used with the frequency domain sensor system, execution of step 1420 may include application of a patient interface to the pregnant mammal's abdomen proximate to a location where the frequency domain sensor system took one or more measurements. The continuous wave sensor system may then be affixed to the patient interface via, for example, an adhesive, a ring like ring 440, an attachment mechanism like attachment mechanisms 340, and/or a track like track 330.

In some embodiments, process 1400 may be executed so that the frequency domain sensor system interrogates maternal tissue above a fetus. One or more results of this interrogation may be used to understand optical properties of the maternal tissue that may be applied to analysis of the continuous wave measurements, which may incident upon and/or pass through both the maternal and fetal tissue. The measurements obtained via execution of step(s) 1410 and/or 1425 may be communicated to, for example, a processor and/or computer 1310 of system 100 for processing to, for example, determine fetal oximetry information and/or characterize maternal and/or fetal tissue by, for example, determining one or more optical properties (e.g., scattering and/or absorption coefficients) thereof according to, for example, one or more processes described herein.

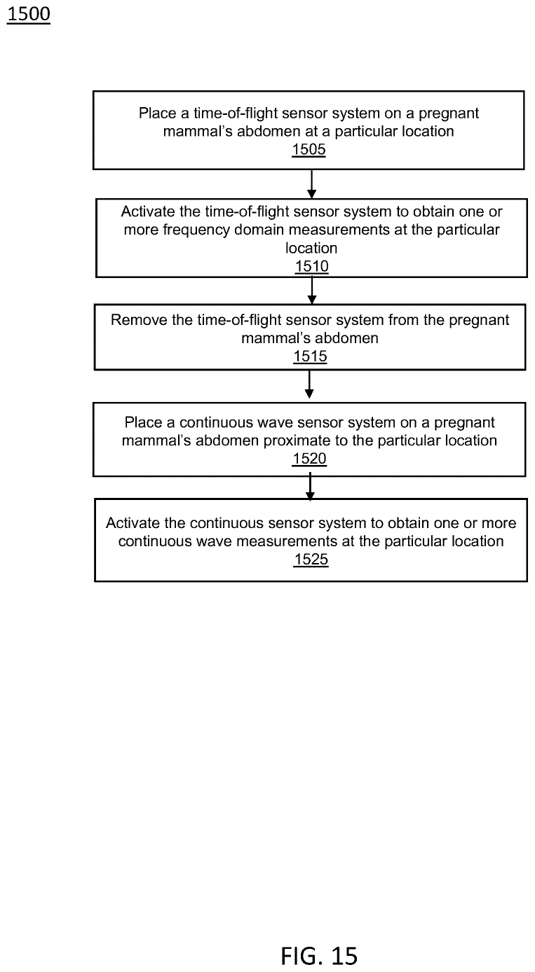

FIG. 15 provides a flowchart illustrating a process 1500 for using a time-of-flight sensor system like time-of-flight sensor system 201 (or components thereof) and a continuous wave sensor system like continuous wave sensor system 301 (or components thereof) to obtain time-of-flight and continuous wave measurements of a pregnant mammal's abdomen and a fetus contained within the pregnant mammal's abdomen. Process 1500 may be executed by, for example, a user such as a clinician or the pregnant mammal using one or more systems and/or devices disclosed herein.

Initially, in step 1505, a time-of-flight sensor system, such as time-of-flight sensor system 201, may be placed on a pregnant mammal's abdomen in a particular location. In some instances, the particular location for placement of the time-of-flight sensor system may be responsive to a position and/or orientation of the fetus within the pregnant mammal's abdomen so that, for example, the time-of-flight sensor system may be placed over a particular portion of the fetus such as the head or back. Fetal position and/or anatomy used to determine a time-of-flight sensor system placement position may be determined via, for example, examination of an ultrasound image and/or a Doppler ultrasound image. In some embodiments, a patient-facing side of the time-of-flight sensor system may be covered with a patient interface that, in some cases, may be removable. Once in position, in step 1510, the time-of-flight sensor system may be activated (e.g., turned on) via, for example, the user to obtain one or more time-of-flight measurements.

In step 1515, the time-of-flight sensor system may be removed from the pregnant mammal's abdomen when, for example, one or more time-of-flight measurements have been taken and a continuous wave sensor system like continuous wave sensor system 301 may then be placed on the pregnant mammal's abdomen at the same position/location from which the time-of-flight sensor system has been removed (step 1520) so that one or more continuous wave measurements may be taken of the pregnant mammal's abdomen and fetus therein when the continuous wave sensor system is activated (step 1525). In some embodiments, a patient-facing side of the continuous wave sensor system may be covered with a patient interface that, in some cases, may be removable prior to execution of step 1520.

Process 1500 may be executed so that the time-of-flight sensor system interrogates the same maternal tissue above a fetus that is interrogated by the continuous wave sensor system so that, for example, the time-of-flight measurements may be used to process the continuous wave sensor system measurements to, for example, isolate a portion of the continuous wave measurement(s) that correspond to light that was incident on the fetus. Additionally, or alternatively, one or more results of analyzing time-of-flight measurements may be to determine one or more optical properties of the maternal tissue that may be applied to analysis of the continuous wave measurements.

In some embodiments, a test to determine whether or not any optical shunting is occurring between components of the time-of-flight sensor system and/or continuous wave sensor system may be performed prior to execution of steps 1510 or 1525, respectively.

In some embodiments, a patient interface such as patient interface 401 and/or an optical blanket such as optical blanket 1210 may be used during execution of process 1500. In some instances, the patient interface may be affixed to the pregnant mammal's abdomen prior to execution of step 1505 and the patient interface may be configured to cooperate with the time-of-flight sensor system via, for example, affixing to the time-of-flight sensor system and/or having one or more windows that correspond to a time-of-flight light source (time-of-flight light source 102) and/or fed detector (e.g., time-of-flight detector 104). Continuous wave sensor system may be affixed to the patient interface via, for example, an adhesive, a ring like ring 440, an attachment mechanism like attachment mechanisms 340, and/or a track like track 330. Then, when time-of-flight sensor system is removed from the pregnant mammal's abdomen (step 1516), the time-of-flight sensor system may be disengaged from the patient interface via, for example, peeling it off an adhesive of the patient interface, disengagement of an attachment mechanism affixing the time-of-flight sensor system to the patient interface, sliding the time-of-flight sensor system out of a track of the patient interface, and/or lifting the time-of-flight sensor system from the ring. Once the time-of-flight sensor system is removed from the patient interface in step 1516, step 1520 may be executed by affixing the continuous wave sensor system to the patient interface via, for example, an adhesive, the ring, the attachment mechanism, and/or the track. In embodiments where a patient interface is not used with the time-of-flight sensor system, execution of step 1520 may include application of a patient interface to the pregnant mammal's abdomen proximate to a location where the time-of-flight sensor system took one or more measurements. The continuous wave sensor system may then be affixed to the patient interface via, for example, an adhesive, a ring like ring 440, an attachment mechanism like attachment mechanisms 340, and/or a track like track 330.

In some embodiments, a test of an optical coupling between time-of-flight sensor system and the pregnant mammal's abdomen may be performed prior to execution of step 1510? to determine whether or not an optical shunting between the time-of-flight light sources and time-of-flight detectors has occurred and, if so, a position and/or optical coupling of time-of-flight sensor system may be adjusted to reduce or eliminate the optical shunting. Additionally, or alternatively, a degree of optical shunting may be determined and then applied to signal(s) received from the time-of-flight sensor system to correct for the optical shunting. Additionally, or alternatively, a test of an optical coupling between the continuous wave sensor system and the pregnant mammal's abdomen may be performed prior to execution of step 1520 to determine whether or not an optical shunting between the continuous wave light sources and continuous wave detectors has occurred and, if so, a position and/or optical coupling of continuous wave sensor system may be adjusted to reduce or eliminate the optical shunting. Additionally, or alternatively, a degree of optical shunting may be determined and then applied to signal(s) received from the continuous wave sensor system to correct for the optical shunting.

In some embodiments, process 1500 may be executed so that the time-of-flight sensor system interrogates maternal tissue above a fetus. One or more results of this interrogation may be used to understand optical properties of the maternal tissue that may be applied to analysis of the continuous wave measurements, which may incident upon both the maternal and fetal tissue. The measurements obtained via execution of step(s) 1510 and/or 1525 may be communicated to, for example, a processor and/or computer 1310 of system 100 for processing to, for example, determine fetal oximetry information and/or characterize maternal and/or fetal tissue by, for example, determining one or more optical properties (e.g., scattering and/or absorption coefficients) thereof according to, for example, one or more processes described herein.

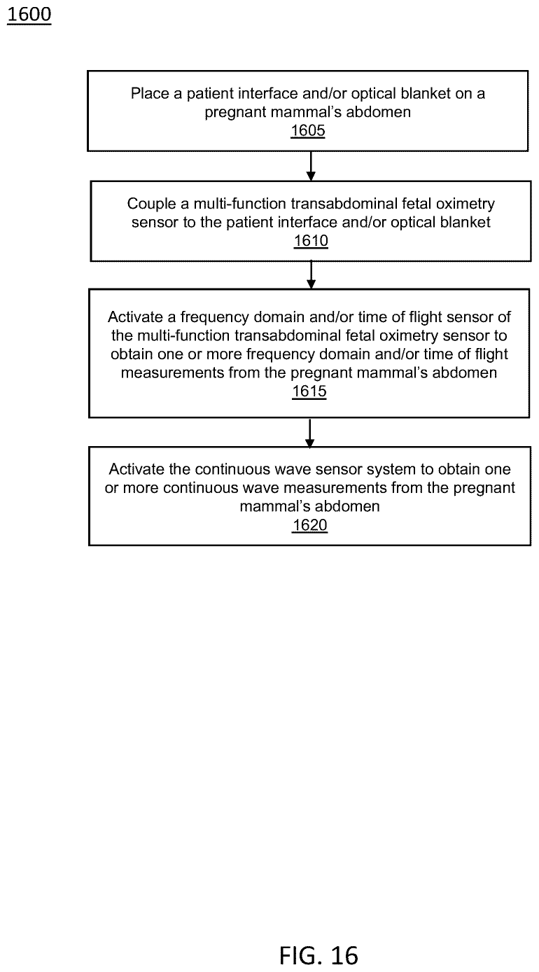

FIG. 16 provides a flowchart illustrating a process 1600 for using a patient interface and a multi-function transabdominal fetal oximetry sensor to obtain one or more transabdominal fetal oximetry measurements of a pregnant mammal's abdomen and a fetus contained within the pregnant mammal's abdomen. Process 1600 may be executed by, for example, a user such as a clinician or the pregnant mammal using one or more systems and/or devices disclosed herein.

In step 1605, a patient interface and/or optical blanket configured to cooperate with a multi-function transabdominal fetal oximetry sensor may be placed and/or affixed on a pregnant mammal's abdomen using, for example, an adhesive compound and/or strap that wraps around the pregnant mammal's abdomen. The multi-function transabdominal fetal oximetry sensor may be, for example, multi-function transabdominal fetal oximetry sensor 501, 502, 600, 901, or 902, the patient interface may be, for example, flexible patient interface assembly 701 or patient interface 1001, 1002, 1200, or 1201. The optical blanket (e.g., optical blanket 1210, 1310, or 1311) may include a hole, or opening, configured to accept insertion of the multi-function transabdominal fetal oximetry sensor therein. Placement of the patient interface and/or optical blanket may be guided by, for example, a position and/or orientation of a fetus, or portion of a fetus (e.g., head or back) within the pregnant mammal's abdomen as may be indicated by, for example, an ultrasound image and/or MRI image of the pregnant mammal's abdomen.

Next, in step 1610, the multi-function transabdominal fetal oximetry sensor may be coupled to the patient interface and/or optical blanket via, for example, aligning one or more components (e.g., patient-facing side of the housing, light source, and/or detector) of the multi-function transabdominal fetal oximetry sensor with the patient interface and/or optical blanket and coupling the multi-function transabdominal fetal oximetry sensor thereto. On some occasions, execution of step 1610 may include affixing a patient-facing side of the multi-function transabdominal fetal oximetry sensor to the patient interface and/or optical blanket via, for example, an adhesive. Additionally, or alternatively, execution of step 1610 may include inserting an alignment and/or attachment mechanism (e.g., attachment mechanism 1060) of the patient interface and/or optical blanket (e.g., attachment mechanism 1330) into a track (e.g., track 1050) of the multi-function transabdominal fetal oximetry sensor. Additionally, or alternatively, execution of step 1610 may include coupling an alignment and/or attachment mechanism (e.g., attachment mechanism 1060) of the patient interface as shown in, for example, FIG. 10C and/or optical blanket (e.g., attachment mechanism 1230) with an attachment mechanism of the multi-function transabdominal fetal oximetry sensor (e.g., attachment mechanism 1055) of the multi-function transabdominal fetal oximetry sensor as shown in, for example, FIG. 12B. Additionally, or alternatively, execution of step 1610 may include inserting the multi-function transabdominal fetal oximetry sensor into a ring of a patient interface like ring 1155 as shown in FIGS. 10D and 12B and/or a ring (e.g., ring 1050) of an optical blanket as shown in FIG. 12D. In some embodiments, a patient-facing side of the multi-function transabdominal fetal oximetry sensor system may be covered with a patient interface that, in some cases, may be removable.

Once the multi-function transabdominal fetal oximetry sensor is in position, a frequency domain sensor system and/or time-of-flight sensor system of the multi-function transabdominal fetal oximetry sensor may be activated to take one or more measurements of the pregnant mammal's abdominal tissue as, for example, described herein (step 1615). Then, in step 1620, the continuous wave sensor system may be activated to obtain one or more continuous wave measurements of the pregnant mammal's abdomen and/or a fetus contained therein. The measurements obtained via execution of step(s) 1615 and/or 1620 may be communicated to, for example, a processor and/or computer 1310 of system 100 for processing to, for example, determine fetal oximetry information and/or characterize maternal and/or fetal tissue by, for example, determining one or more optical properties (e.g., scattering and/or absorption coefficients) thereof according to, for example, one or more processes described herein.

In some embodiments, a test of an optical coupling between the multi-function transabdominal fetal oximetry sensor and the pregnant mammal's abdomen may be performed prior to execution of step 1615 to determine whether or not an optical shunting between the light sources and detectors of the multi-function transabdominal fetal oximetry sensor has occurred and, if so, a position and/or optical coupling of multi-function transabdominal fetal oximetry sensor may be adjusted to reduce or eliminate the optical shunting. Additionally, or alternatively, a degree of optical shunting may be determined and then applied to signal(s) received from the multi-function transabdominal fetal oximetry sensor to correct for the optical shunting.

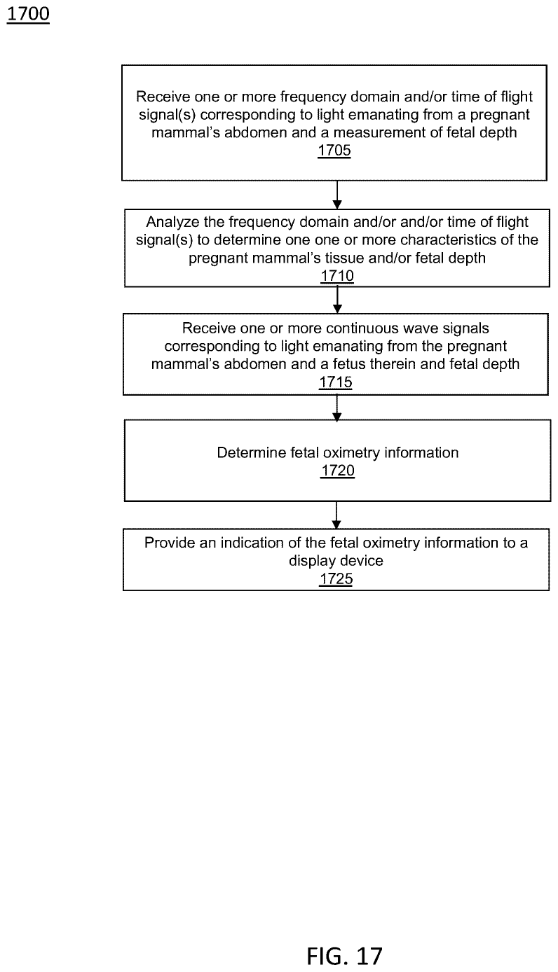

FIG. 17 provides a flowchart illustrating a process 1700 for determining fetal oximetry information using measurements from, for example, one or more of the frequency domain sensor system, time-of-flight sensor system, continuous wave sensor system, and/or multi-function transabdominal fetal oximetry sensor disclosed herein. Process 1700 may be executed by, for example, system 100, controller system 540, and/or any component thereof.

Initially, in step 1705, one or more frequency domain and/or time-of-flight signal(s) corresponding to light emanating from a pregnant mammal's abdomen may be received from, for example, a frequency domain detector (e.g., frequency domain detector 104) and/or a time-of-flight detector (e.g., time-of-flight detector 204) of frequency domain sensor system 101, time-of-flight sensor system 201, multi-function transabdominal fetal oximetry sensor 501, or multi-function transabdominal fetal oximetry sensor 502. Alternatively, the signal(s) received in step 1705 may be received from first frequency domain detector 935A and a second frequency domain detector 935B of multi-function transabdominal fetal oximetry sensor 901 or multi-function transabdominal fetal oximetry sensor 902. The light emanating from the pregnant mammal's abdomen may correspond to light emitted by a frequency domain light source (e.g., frequency domain light source 102) and/or a time-of-flight light source (e.g., time-of-flight light source 202). Alternatively, the light emanating from the pregnant mammal's abdomen may correspond to light emitted by a frequency domain light source 930 of multi-function transabdominal fetal oximetry sensor 901 or multi-function transabdominal fetal oximetry sensor 902. Optionally, position information for one or more components (e.g., light source and/or detector) generating the signal(s) received in step 1705 may also be received in step 1705. In some embodiments, a measurement of fetal depth (e.g., a distance between the pregnant mammal's abdomen and the fetus' epidermis) may be received in step 1705. The fetal depth may be measured using, for example, one or more ultrasound images of the pregnant mammal's abdomen at the same site from which the frequency domain and/or time-of-flight signal(s) are received so that, for example, a depth of the fetus at the particular location from which the frequency domain and/or time-of-flight signal(s) are obtained is known.

The one or more frequency domain and/or time-of-flight signal(s) corresponding to light emanating from a pregnant mammal's abdomen may then be analyzed to determine one or more characteristics of the maternal tissue (step 1710). Exemplary characteristics include, but are not limited to, width, tissue type, how the maternal tissue scatters light, and/or how the maternal tissue absorbs light. In some embodiments, the analysis of step 1710 may further include using fetal depth to determine one or more characteristics of the maternal tissue.

In step 1715, one or more continuous wave signal(s) corresponding to light emanating from a pregnant mammal's abdomen may be received from, for example, a continuous wave detector (e.g., continuous wave detector 304) of continuous wave sensor system 301, multi-function transabdominal fetal oximetry sensor 510, or multi-function transabdominal fetal oximetry sensor 520 or a continuous wave detector 920 of multi-function transabdominal fetal oximetry sensor 901 or 902. The light emanating from the pregnant mammal's abdomen may correspond to light emitted by, for example, a continuous wave light source from a continuous wave sensor system such as continuous wave light source 302 of continuous wave sensor system 301 or optical source 940 of multi-function transabdominal fetal oximetry sensor 901 or 902. In many embodiments, the frequency domain and/or continuous wave signal(s) of step 1705 and the continuous wave signal(s) of step 1715 may be received from the same location on the pregnant mammal's abdomen and/or positions on the pregnant mammal's abdomen that are proximate (e.g., a separation of 0.2-5 cm) to one another. In some embodiments, a measurement of fetal depth (e.g., a distance between the pregnant mammal's abdomen and the fetus'epidermis) at a position on the maternal abdomen from which the continuous wave measurements are taken may be received in step 1715.

In step 1720, the continuous wave signal(s) and optional fetal depth received in step 1715 may be analyzed using the frequency domain signal(s) and/or time-of-flight signal(s) received in step 1705 and/or the characteristics of the pregnant mammal's tissue determined in step 1710 may be used to determine fetal oximetry information for the pregnant mammal's fetus. In some embodiments, the frequency domain signal(s) and/or time-of-flight signal(s) received in step 1705 and/or characteristics of the pregnant mammal's tissue determined in step 1710 may be used to reduce and/or eliminate maternal contributions to the continuous wave signal(s) received in step 1715, which may serve to, for example, isolate fetal contributions to the continuous wave signal(s) so that they may be further analyzed to determine fetal oximetry information. These fetal contributions to the continuous wave signal(s) may then be analyzed and/or processed to determine and/or calculate fetal oximetry information via, for example, application of the Beer Lambert law or variations thereof. Following step 1720, an indication of the fetal oximetry information may be provided to a display device (e.g., display device 1315) (step 1725).

In some embodiments, a test of an optical coupling between the multi-function transabdominal fetal oximetry sensor and the pregnant mammal's abdomen may be performed prior to execution of step 1705 to determine whether or not an optical shunting between the light sources and detectors of the multi-function transabdominal fetal oximetry sensor has occurred and, if so, a position and/or optical coupling of the multi-function transabdominal fetal oximetry sensor may be adjusted to reduce or eliminate the optical shunting. Additionally, or alternatively, a degree of optical shunting may be determined and then applied to signal(s) received from the multi-function transabdominal fetal oximetry sensor to correct for the optical shunting.

Additionally, or alternatively, execution of some, or all, steps of process(es) 1400, 1500, 1600, and/or 1700 may be repeated on, for example, an as-needed and/or periodic basis (e.g., every 5, 10, 15, 20, 40, 45, 60 or 120) minutes. Repetition of some, or all, steps of process(es) 1400, 1500, 1600, and/or 1700 on an as-needed basis may be responsive to, for example, an error, an unexpected measurement result, and/or a changing position of the fetus within the pregnant mammal's abdomen and/or birth canal during labor and delivery. For example, in some situations, steps 1405-1425, 1505-1525, 1605-1620, and/or 1715-1725 may be repeated on a continuous or relatively frequent periodic basis (e.g., every 10-60, every 1-15 minutes) to, for example, monitor fetal oximetry values and steps 1705 and 1710 may be repeated on less frequent basis (e.g., every 5-60 minutes, every 1-7 hours) to, for example, confirm characterizations of maternal optical characteristics have not changed and/or update them when they have. In some embodiments, a rate of repetition of execution of process(es) 1400, 1500, 1600, and/or 1700 may be responsive to physiological conditions (e.g., greater frequency for a complex labor and delivery) and/or a stage of labor and delivery for the pregnant mammal and/or fetus. For example, as the fetus moves through the birth canal during delivery (stage 2 of labor), its position within the pregnant mammal's abdomen changes more quickly than during the first stage of labor. Thus, steps 1405 and 1410; 1505 and 1510; 1605 and 1610; and/or 1705 and 1710 may be performed more frequently during the second stage of labor because the fetal depth and/or optical characteristics of the pregnant mammal's abdomen in relation to the fetus will change more rapidly during the second stage of labor than during the first stage of labor and the determined characteristics and/or fetal depth will need to be updated accordingly so that accuracy of the fetal oximetry determination (step 1720) is maintained and/or the continuous wave measurements received following activation in steps 1425, 1525, and/or 1620 are properly analyzed using the time-resolved (e.g., frequency domain and/or time-of-flight) measurements.

We claim:

1. A method for transabdominally determining fetal oximetry information, the method comprising:
receiving, by a processor, a time resolved signal corresponding to emanating from a pregnant mammal's abdomen;
determining, by the processor, a characteristic of the pregnant mammal's tissue using the received time resolved signal;
receiving, by the processor, one or more continuous wave signals corresponding to light emanating from the pregnant mammal's abdomen;
determining, by the processor, fetal oximetry information using the one or more continuous wave signals and the characteristic of the pregnant mammal's tissue; and
providing, by the processor, the fetal oximetry information to a display device.

2. The method of claim 1, further comprising:
receiving, by the processor, a measurement of a depth of the fetus at a position corresponding to a component of the time resolved sensor system, wherein determining the characteristic of the pregnant mammal's tissue further uses the received measurement of fetal depth.

3. The method of claim 1 or 2, further comprising:
receiving, by the processor, a measurement of a depth of the fetus at a position corresponding to a component of the continuous wave sensor system, wherein determining the fetal oximetry information further uses the received measurement of fetal depth.

4. The method of any of claims 1-3, wherein the fetal oximetry information is at least one of a fetal hemoglobin oxygen saturation level and a fetal tissue oxygenation level.

5. The method of any of claims 1-4, wherein the time resolved signal corresponds to light of a wavelength greater than 805 nm and light of a wavelength less than 805 nm.

6. The method of any of claims 1-5, wherein one or more continuous wave signals include a first continuous wave signal that corresponds to light of a wavelength greater than 805 nm and a second continuous wave signal that corresponds to light of a wavelength less than 805 nm.

7. The method of any of claims 1-6, wherein a position of the time resolved sensor system on the pregnant mammal's abdomen when the light emanates from the pregnant mammal's abdomen and is detected by the time resolved sensor system is proximate to a position of the continuous wave measurement device on the pregnant mammal's abdomen when the light emanates from the pregnant mammal's abdomen and is detected by the continuous wave measurement device.

8. The method of any of claims 1-7, wherein a position of the time resolved sensor system on the pregnant mammal's abdomen when the light emanates from the pregnant mammal's abdomen and is detected by the time resolved sensor system is the same as the position of the continuous wave measurement device on the pregnant mammal's abdomen when the light emanates from the pregnant mammal's abdomen and is detected by the continuous wave measurement device.

9. The method of any of claims 1-8, wherein the time resolved sensor system interrogates maternal tissue through which light emitted by the continuous wave measurement device passes.

10. The method of any of claims 1-9, wherein the time resolved sensor system interrogates maternal tissue proximate to maternal tissue through which light emitted by the continuous wave measurement device passes.

11. The method of any of claims 1-10, further comprising:
determining a characteristic of the fetal tissue.

12. The method of any of claims 1-11, wherein the continuous wave sensor system interrogates fetal tissue through which light emitted by the continuous wave measurement device passes.

13. The method of any of claims 1-12, wherein the time resolved sensor system is at least one of a frequency domain sensor system and a time-of-flight sensor system.

14. A system comprising:
a processor in communication with a memory; and
the memory, the memory being configured to store a set of instructions thereon,
which when executed by the processor cause the processor to execute any of the methods of claims 1-13.

15. A patient interface configured to cooperate with a transabdominal sensor that includes a light source and a plurality of detectors, each detector of the plurality of detectors including a light detecting surface, the patient interface comprising:
a sheet of flexible material, the sheet comprising:
a plurality of detector openings, a size and position of each detector opening of the plurality of detector openings corresponding to a size and position of a light detecting surface of a respective one of the plurality of detectors; and
a light source opening, a size and position of the light source opening corresponding to a size and position of the light source.

16. The patient interface of claim 15, wherein the transabdominal sensor is a transabdominal fetal oximetry sensor.

17. The patient interface of claim 15 or 16, wherein the transabdominal sensor is at least one of a frequency domain sensor system, a time-of-flight sensor system, a continuous wave sensor system, and a multi-function transabdominal fetal oximetry sensor.

18. The patient interface of any of claims 15-17, further comprising:
a ring configured to align with an outer edge of the patient interface and fit over an outer edge of a patient-facing side of the transabdominal sensor, thereby preventing ambient light from entering a side of the transabdominal sensor when positioned on a pregnant mammal's abdomen.

19. The patient interface any of claims 15-18, further comprising:
an adhesive configured to stick to a pregnant mammal's abdomen.

20. The patient interface of any of claims 15-19, wherein at least one of the detector openings is covered with a transparent material in a watertight manner.

21. The patient interface of any of claims 15-20, wherein at least one of the detector openings are covered with a material that filters light of undesired wavelengths.

22. The patient interface of any of claims 15-21, wherein the sheet of flexible material is configured to form a tight optical couple between a pregnant mammal's abdomen and the transabdominal sensor.

23. The patient interface of any of claims 15-22, wherein the sheet of flexible material is configured to prevent shunting light from an optical source of the transabdominal sensor directly to a detector of the transabdominal sensor without passing through tissue of a pregnant mammal's abdomen on which the transabdominal sensor is positioned.

24. The patient interface of any of claims 15-23, wherein the patient interface is configured to cooperate with the transabdominal sensor by being removably affixed thereto.

25. The patient interface of any of claims 15-24, wherein the patient interface is configured to be removably attached to transabdominal sensor and to remain on a pregnant mammal's abdomen when the transabdominal sensor is removed from the patient interface.

26. The patient interface of any of claims 15-25, wherein the patient interface further includes an optical blanket configured to cover a portion of a pregnant mammal's abdomen proximate to the transabdominal sensor and prevent ambient light from entering the covered portion of the pregnant mammal's abdomen.

27. The patient interface of claim 26, wherein the optical blanket includes an opening sized, shaped, arranged, and configured to cooperate with the patient interface.

28. The patient interface of claim 26 or 27, wherein the optical blanket includes an attachment mechanism configured to attach to the transabdominal sensor.

29. The patient interface of any of claims 15-28, wherein the transabdominal sensor is configured to transabdominally obtain optical signals that may be processed to determine transabdominal fetal oximetry information.

30. A method of using a patient interface comprising:
affixing a patient interface to an epidermis of a pregnant mammal's abdomen above a fetus positioned within, the patient interface including a sheet of flexible material that includes a light source opening and a plurality of detector openings; and
positioning a transabdominal sensor that includes a light source and a plurality of detectors proximate to the patient interface so that the light source aligns with the light source opening in the patient interface and each of the plurality of detectors aligns with a respective detector window of the patient interface.

31. The method of claim 30, further comprising attaching the transabdominal sensor to the patient interface.
